SEQUENCE LISTING (1 sequence)

SEQ ID NO: 1 - protein, 10 aa, Artificial Sequence
  stability sequence
  VARIANT 3, 4, 5, 6: Xaa = any amino acid
MGXXXXGGPP

(19) United States
(12) Patent Application Publication
(10) Pub. No.: US 2007/0184487 A1
(43) Pub. Date: Aug. 9, 2007

(54) Compositions and methods for design of non-immunogenic proteins

(76) Inventor: Brian M. Baynes, Cambridge, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/485,786

(22) Filed: Jul. 12, 2006

Related U.S. Application Data
(60) Provisional application No. 60/698,319, filed on Jul. 12, 2005.

(51) Int. Cl. C40B 30/06; C40B 40/10; A61K 38/10; A61K 38/08; C07K 7/08; C07K 7/06

(52) U.S. Cl. 435/7.1; 435/69.1; 514/15; 514/16; 514/17; 530/328; 530/329; 530/330

(57) ABSTRACT

Provided are methods for de novo design of proteins that are non-immunogenic when administered for therapeutic purposes. The methods involve protein design based on combinations of peptide fragments naturally encountered by the immune system.

### RELATED APPLICATIONS

This application claims the benefit of U.S. Provisional Application No. 60/698,319, filed Jul. 12, 2005, which application is hereby incorporated by reference in its entirety.

### BACKGROUND OF THE INVENTION

The problem of immunogenicity plagues therapeutic protein design. Fear of immune response leads protein designers to minimize the number of changes made to a protein from a fully human reference sequence. In practice, a balance must be struck between the extent of improvement of a target property (e.g. potency, binding affinity, or catalytic efficiency) and the number of changes made. Thus, the final engineered protein is often very close to the initial protein in sequence space. Alternatively, as in the case of monoclonal antibodies, designers attempt to “humanize” a therapeutic protein by creating chimeric proteins having largely human structures in the hopes of thwarting the human immune recognition. These conventional approaches are far from optimal, and confine a protein designer to a limited range of alterations that may not include optimally active or stable therapeutic proteins.

The immune system does not examine each protein in its entirety. Rather, the immune system is regulated by peptides derived from a protein which has been processed, or digested, within immune cells. These peptides are subsequently presented on the surface of the immune cells for recognition by T cells. It is these presented peptides that elicit an immune response. Thus, if these peptides are recognized as self (e.g., as native to the host), the protein from which these peptides were derived is non-immunogenic.

Given the limitations of conventional approaches for protein design, new methods are needed for designing and producing non-immunogenic proteins based on an understanding of immune system stimulation. Non-immunogenic proteins produced by such methods are also desirable.

### BRIEF SUMMARY OF THE INVENTION

The present invention provides compositions and methods for designing proteins having one or more desired characteristics and low, or no, immunogenicity in a host, such as a human. Based on an understanding of peptide presentation by the immune system, a non-immunogenic protein may be designed such that it will degrade into peptides that are similar to, or the same as, peptides generated by degradation of native human proteins. However, the designed protein will comprise a sequence that is not found in the native complement of human proteins.

In one aspect, the invention provides a library of sequences of peptide motifs found in human proteins. The library comprises a plurality of sequences of human peptides of a given size range, having more than 4 amino acid residues, preferably more than 5, 6, 7, 8, 9, or 10 amino acid residues, and less than about 50 amino acid residues, preferably less than about 40, 30, 20, or 15 amino acid residues. In one embodiment, the library comprises sequences of peptides having about 6 to 15 amino acid residues, about 8 to 12 amino acid residues, or about 8 to 10 amino acid residues. The library may also include information about the geometries and conformations that these peptides may assume, such as alpha helix, beta sheet, random coil, and disordered region. The library may optionally include additional information about the conformation such as the relative positions of the alpha carbons of the peptide backbone.

In a preferred embodiment, the library comprises sequences of peptides that are produced when a human protein is processed for antigen presentation. Thus, peptide sequences represent peptides that would be produced upon protein processing by cellular machinery, such as, for example, digestion by proteasomes in the cytosol, or acid protease cleavage in the intracellular vesicles of macrophages, immature dendritic cells, B cells, or other antigen-presenting cells. In one embodiment, the library of sequences of peptide motifs comprises those peptide motifs that are generated using proteasome or acid protease cleavage sites of the peptide sequences from naturally occurring human proteins. In another embodiment, the library of sequences of peptide motifs comprises those of peptides presented by the Major Histocompatibility Complex I or II on the surface of human immune cells.

In another aspect, the invention provides a library of sequences of peptide motifs found in human proteins, wherein the human proteins are members of a distinct class of molecules, said class defined by a structural motif or function.

In another aspect, the invention provides a library comprising isolated polynucleotides encoding a set of all human peptide sequences having more than 4 amino acid residues, and less than about 50 amino acid residues.

In another aspect, the invention provides a library comprising polynucleotides encoding peptide motifs found in human proteins, wherein the human proteins are members of a distinct class of molecules, said class defined by a structural motif or a function.

In another aspect, the invention provides a biosynthetic library comprising a plurality of synthetic DNAs of known and planned, as opposed to randomized, sequence. The library comprises polynucleotides encoding peptides of the peptide library, which can be selected or screened for species having a predetermined property or set of properties, or may be selected or screened themselves for polynucleotides having particular functional or structural properties. The polynucleotides in the libraries preferably are chemically synthesized or are assembled from chemically synthesized oligonucleotides using techniques such as those set forth herein. The plural polynucleotides of the library may comprise regions of significant sequence homology. Alternatively, or in addition, the library members may have reading frames exploiting consistent codon usage patterns so as to promote similar expression levels in a selected cellular or cell free expression system, e.g., a ribosomal expression system, a phage expression system, or an E. coli expression system. Preferably, the oligonucleotides are synthesized in parallel. It is also preferred to assemble the polynucleotides in parallel from the chemically synthesized oligonucleotides.

In another aspect, the invention provides a method of designing a protein using a peptide sequence library described herein. In exemplary embodiments, the protein has reduced immunogenicity as compared to a reference protein or is non-immunogenic for a desired host. Using known methods of computational or in silico protein design, a person skilled in the art will be able to design a protein de novo, or modify a starting protein, by choosing one or more peptides from the library. For example, the structure of a known protein may be used to identify one or more members of the peptide library that have a structure which closely resembles a portion of the known protein. Structural similarity between a portion of the protein and a peptide in the library may be identified by overlaying the three-dimensional peptide structure onto a domain, a motif, or any partial structure of the protein. Thus, a new protein may be designed by replacing at least one original part of the structure of a known protein with a member of the peptide library. One or more parts of the known protein can be replaced. In certain embodiments, all possible combinations of two or more peptides from the library can be made in silico to produce a library of hypothetical new proteins. Following the creation of the library of such new proteins, each protein as a whole can be computationally evaluated for one or more properties of interest.

In another aspect, the invention provides a method for producing a protein having one or more desired characteristics or properties comprising: generating sequence data for a plurality of possible proteins using the peptide library described above; in parallel, assembling a plurality of polynucleotides that encode at least 10 of the proteins; expressing the proteins from the polynucleotides; and selecting or screening the proteins to identify proteins having one or more desired characteristics using a high throughput assay. A preferred method for assembly the polynucleotides involves assembling construction oligonucleotides by hybridization of complementary, overlapping oligonucleotide sequences followed by ligase and/or polymerase treatment, to produce at least 20, 50, 100, 10³, 10⁴, 10⁵, or 10⁶ of the sequences of the proteins. Alternatively, oligonucleotides encoding each peptide sequence of the library described above, along with appropriate junction oligonucleotides, could be made, assembled with PCR into a combinatorial library and translated to produce a protein library. The proteins may then be assayed for the desired function or property, using assays known for such function or property. Alternatively, the methods may involve construction of large polynucleotides with high fidelity using stepwise assembly of complementary, overlapping, oligonucleotides. In exemplary embodiments, at least 10, 100, 1,000, 10,000, 100,000 or more designed proteins are experimentally tested. Once a desired protein is identified, it may be produced in useful quantities by any method known in the art. In a preferred embodiment, the production process does not comprise post-translational modifications that may introduce one or more moieties that are immunogenic in humans. Examples of post-translation modifications include, for example, glycosylation, acylation, phosphorylation, methylation, sulfation and prenylation.

In some embodiments, initial screening may be carried out in silico, wherein the predicted structures of the proteins assembled from the peptide sequences in the library are compared with a naturally occurring protein having one or more desired characteristics. Library proteins sharing structural elements that correlate with a desired characteristic of the naturally occurring protein are selected as candidate proteins. These candidate proteins are then expressed from synthetic polynucleotides and tested for the desired characteristic. Proteins exhibiting a desired characteristic may be selected and produced as described above.

In another aspect, the invention provides proteins designed and manufactured using the sequences of the peptide library and the methods described above. The designed proteins may be produced by any means known in the art, including peptide synthesis or expression from recombinant DNA molecules. In addition to a desired therapeutic functionality, a designed protein of the present invention may be non-immunogenic or have low immunogenecity in humans. In certain embodiments, the designed proteins may be free of posttranslational modifications. In other embodiments, the designed protein may only comprise posttranslational modifications that are non-immunogenic in humans, for example, by being identical to post translational modifications naturally occurring in humans.

In another aspect, the invention provides a method of designing a novel protein comprising: (a) selecting a scaffold protein; (b) identifying a partial structure of the scaffold protein to be replaced; (c) computationally searching and identifying a human peptide, wherein the human peptide: (i) is a member of a library comprising a set of all sequences of human peptides having more than 4 amino acid residues and less than about 50 amino acid residues; and (ii) shares a structural motif with the partial structure of the scaffold protein; (d) replacing a portion of the amino acid sequence of the scaffold protein corresponding to the partial structure with the amino acid sequence of the human peptide to produce a novel protein; and (e) optimizing the structure of the novel protein to retain the structural motif.

In another aspect, the invention provides a method of producing a novel protein, comprising: (a) selecting a scaffold protein; (b) identifying a partial structure of the scaffold protein to be replaced; (c) computationally searching and identifying one or more human peptides, wherein the human peptides: (i) are a member of library comprising a set of all sequences of human peptides having more than 4 amino acid residues and less than about 50 amino acid residues; and (ii) share a structural motif with the partial structure; and (d) replacing the partial structure sequence with the sequence of a human peptide to create a sequence of the novel protein; (e) creating a polynucleotide that encodes the amino acid sequence of the novel protein; and (f) expressing the polynucleotide to produce the novel protein.

In one embodiment, the invention provides a library of novel proteins, wherein the novel proteins are produced by a method described herein, and wherein the novel proteins are non-immunogenic in humans. In another embodiment, the invention provides a method for producing a therapeutic, non-immunogenic protein comprising screening a library of novel proteins produced by a method described herein to identify a protein exhibiting a desired characteristic. In another embodiment, the invention provides a protein produced by the methods described herein.

In another aspect, the invention provides a protein which is non-immunogenic to humans, wherein the protein comprises human peptide segments, which peptide segments are recognized as self by the human immune system, and wherein the protein does not naturally occur in humans.

In another aspect, the invention provides a pharmaceutical composition comprising: (a) an isolated and purified protein comprising human peptide segments, which peptide segments are recognized as self by the human immune system, and wherein the protein does not naturally occur in humans; and (b) a pharmaceutically acceptable excipient.

In another aspect, the invention provides a method of designing a novel protein comprising: (a) selecting a scaffold protein; (b) identifying a partial structure or disordered region of the scaffold protein to be replaced; (c) computationally searching and identifying one or more human peptides, wherein the human peptides: (i) are a member of a library comprising a set of all sequences of human peptides having more than 4 amino acid residues and less than about 50 amino acid residues; and (ii) share a structural motif with the partial structure of the scaffold protein or are disordered; (d) replacing a portion of the amino acid sequence of the scaffold protein corresponding to the partial structure or disordered region with the amino acid sequence of a human peptide to produce a novel protein; and (e) optimizing the structure of the novel protein to retain the overall structure of the scaffold protein.

In certain embodiments, the methods described herein may further comprise (i) creating a polynucleotide that encodes the amino acid sequence of the novel protein, and/or (ii) expressing the polynucleotide to produce the novel protein.

In certain embodiments, the novel proteins produced by the methods described herein are non-immunogenic in humans.

In certain embodiments, the invention provides a library of novel proteins, wherein the novel proteins are produced by a method described herein. In other embodiments, such libraries may be screened so as to produce a therapeutic, non-immunogenic protein exhibiting a desired characteristic.

The practice of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, 3rd Ed., (Sambrook and Russell eds., Cold Spring Harbor Laboratory Press: 2001); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells: A Manual of Basic Technique, 4th Ed. (R. I. Freshney, Wiley-Liss, 2000); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); Current Protocols in Molecular Biology, (Brent et al. eds. John Wiley & Sons Inc., 2003); Current Protocols in Immunology (J. E. Coligan, et al. eds., John Wiley & Sons Inc., 1993).

Other features and advantages of the invention will be apparent from the following detailed description, and from the claims. Although the descriptions are for designing proteins that are non-immunogenic to humans, the same principle applies to designing proteins that are non-immunogenic to any other vertebrates, including mammals such as mouse, rat, rabbit, dog, cat, horse, bovine, sheep, pig, or monkey.

The claims provided below are hereby incorporated into this section by reference.

### BRIEF DESCRIPTION OF THE DRAWINGS

FIGS. 1A-1C. Simplified illustration of an example DNA molecule to be synthesized.

FIG. 2. Illustrates a microarray used in the synthesis of the exemplary DNA molecule of FIG. 1.

FIG. 3. Removal of error sequences using mismatch binding proteins.

FIG. 4. Neutralization of error sequences with mismatch recognition proteins.

FIG. 5. Strand-specific error correction.

FIG. 6. Local removal of DNA on both strands at the site of a mismatch.

FIG. 7. Another scheme for local removal of DNA on both strands at the site of a mismatch.

FIG. 8. Summarizes the effects of the methods of FIG. 6 (or equivalently, FIG. 7) applied to two DNA duplexes, each containing a single base (mismatch) error.

FIG. 9. Shows an example of semi-selective removal of mismatch-containing segments.

FIG. 10. Shows a procedure for reducing correlated errors in synthesized DNA.

FIG. 11. Illustrates possible crossover products that may arise when assembling nucleic acid species containing homologous regions.

FIG. 12. Illustrates crossover polymerization that may occur when assembling nucleic acid species with internal homologous regions.

FIG. 13. Illustrates the circle selection method for removal of undesired crossover products.

FIG. 14. Illustrates one embodiment of the size selection method for removal of undesired crossover products.

FIG. 15. Illustrates another embodiment of the size selection method for removal of undesired crossover products.

### DETAILED DESCRIPTION OF THE INVENTION

1. Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

The term “amplification” means that the number of copies of a nucleic acid fragment is increased.

The term “characteristic,” as used herein with reference to a protein or protein variant, refers to a biochemical and/or biophysical property of a protein. Examples of biophysical properties, include for example, thermal stability, solubility, isoelectric point, pH stability, crystalizability, conditions of crystallization, aggregation state, heat capacity, resistance to chemical denaturation, resistance to proteolytic degradation, amide hydrogen exchange data, behavior on chromatographic matrices, electrophoretic mobility, resistance to degradation during mass spectrometry, and results obtained from nuclear magnetic resonance, X-ray crystallography, circular dichroism, light scattering, atomic adsorption, fluorescence, fluorescence quenching, mass spectroscopy, infrared spectroscopy, electron microscopy, and/or atomic force microscopy. Examples of biochemical properties include, for example, expressability, protein yield, small-molecule binding, subcellular localization, utility as a drug target, protein-protein interactions, and protein-ligand interactions.

The term “cleavage” as used herein refers to the breakage of a bond between two nucleotides, such as a phosphodiester bond, or the breakage of a peptide bond between two adjacent amino acids.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term “domain” refers to a unit of a protein or protein complex, comprising a polypeptide subsequence, a complete polypeptide sequence, or a plurality of polypeptide sequences where that unit has a defined function. The function is understood to be broadly defined and can include for example, ligand binding, catalytic activity or structure stabilization of the protein.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “heterologous,” as used herein in the context of a chimeric polynucleotide, refers to sequences comprising segments, domains, or genetic elements, the exact combination and sequence of which is not found in nature.

The term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double-helix, i.e. where the ligation process ligates a “nick” at a ligatable nick site and creates a complementary duplex (Blackburn, M. and Gait, M. (1996) in Nucleic Acids in Chemistry and Biology, Oxford University Press, Oxford, pp. 132-33, 481-2). The site between the adjacent oligonucleotides is referred to as the “ligatable nick site”, “nick site”, or “nick”, whereby the phosphodiester bond is non-existent, or cleaved.

The term “ligate” refers to the reaction of covalently joining adjacent oligonucleotides through formation of an internucleotide linkage.

The term “motif” refers to an amino acid sequence that is commonly found in a protein of a particular structure or function. Typically, a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases may be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The term “mutations” means changes in the sequence of a wild-type nucleic acid or polypeptide sequence. Such mutations may be point mutations such as transitions or transversions. The mutations may be deletions, insertions or duplications.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

“Polypeptide” and “peptide” are used interchangeably herein to refer to a polymer of amino acid residues; whereas a “protein” typically contains one or multiple polypeptide chains. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “residue,” as it relates to a polynucleotide, refers to either a purine or pyrimidine nucleotide; as it relates to a polypeptide, it refers to an amino acid.

The term “structural motif”, when used in reference to a polypeptide, refers to a secondary or tertiary structure that may be shared by a variety of polypeptides having different amino acid sequences. For example, certain amino acid residues within a motif, or alternatively their backbone or side chains (which may or may not include the Cα atoms of the side chains) are positioned in a like relationship with respect to one another in the motif.

The term “wild-type” means that the nucleic acid fragment or polypeptide does not comprise any mutations. A “wild-type” protein means that the protein will be active at a comparable level of activity found in nature and typically will comprise an amino acid sequence found in nature. In an aspect of the invention, the term “wild type” or “parental sequence” can indicate a starting or reference sequence prior to manipulation of the sequence.

2. Overview

The immune system does not examine each protein in its entirety. Rather, the immune system is regulated by peptides derived from a protein which has been processed, or digested, within immune cells. The peptides are subsequently presented on the surface of the immune cells for recognition by T cells. It is these presented peptides that elicit an immune response. If these peptides are recognized as having come from the host (e.g., self), the protein from which these peptides were derived is non-immunogenic. Based on an understanding of peptide presentation by the immune system, proteins may be designed to avoid or reduce an immunogenic response. For example, proteins may be designed to degrade into peptides that are similar to, or the same as, peptides produced upon degradation of native human proteins. Using the methods described herein, proteins may be designed that have non-wild-type sequences but which degrade into peptides recognized by the host immune system as self peptides.

De novo protein design methodologies have become significantly more powerful in the past decade. It is now possible to screen libraries of >10¹⁰⁰ protein sequences in silico, not by computationally checking each one, but rather by exploiting an algorithm to eliminate certain regions of sequence space. See Design of a Novel Globular Protein Fold with Atomic Level Accuracy, Kuhlman et al., Science, V203, p. 1344, 2003. These library sizes are staggering in comparison with experimental methods, which top out at library sizes of about 10¹² to 10¹⁵.

The caveat of in silico methods is that they rely heavily on empirical models of protein function, and thus, currently have far less than perfect accuracy. To compensate for model inaccuracies, the output of in silico models is generally a rank-ordered list of possible designs, where each design is assigned a score. One then ends up with a list of “highly likely solutions” at the top of this ordered list, some subset of which can be synthesized or mutated from wild type sequences and tested. Still, this approach has had some notable successes including, for example, design of a novel 27 amino acid sequence αββ motif with a predefined backbone (Dahiyat and Mayo 1997, Science 278: 82-87), design of a novel iron superoxide dismutase (Pinto et al. 1997, Proc. Natl. Acad. Sci. USA 94: 5562-5567), design of a novel 93 amino acid protein fold not found in nature, “Top7” (Kuhlman et al. 2003, Science 302: 1364-1368), addition of enzymatic activity (triose phosphate isomerase) into a nonenzyme scaffold (ribose binding protein) using protein design (Dwyer et al. 2003, Science 304: 1967-1971), design of novel sensor proteins (Looger et al. 2003, Nature 423: 185-190), and design of a therapeutic protein variant (dominant negative TNF-alpha variant) (Steed et al 2003, Science 301: 1895-1898).

The field is becoming increasingly aware that the empirical models used to score each design may not be sufficiently good to separate the best 10 or 20 designs from the others. This was highlighted in a recent paper pointing out how some models are used to make predictions far from their optimal regimes (Jaramillo and Wodak 2005, Biophys. J. 88: 156-171). Practitioners have a desire to synthesize and test more than ˜10 of their in silico designs, perhaps 100 to 1000, or even 10000, proteins instead, to avoid missing possible solutions to the design problem due to only a slight error in the model. Methods for synthesizing a large number of polynucleotides at low cost is described in U.S. Provisional Application No. 60/643,813, filed Jan. 13, 2005, the disclosure of which is incorporated by reference herein. These methods enable protein designers to build, at reasonable cost and in a reasonable time, a far greater portion (or all) of their high scoring designs, perhaps 10⁴ specific sequences or more.

In one embodiment, the invention provides methods for producing libraries of novel proteins produced from peptide sequences derived from human proteins. In an exemplary embodiment, the library proteins are designed using one or more peptides representing the natural degradation products of human proteins by the host immune system, e.g., peptides that are recognized as self by the host. The library may be produced using de novo protein design or by modifying known proteins to create new proteins having one or more desired characteristics, such as biological activities or stability. In exemplary embodiments, the designed proteins will be non-immunogenic or have low immunogenecity in a host, such as, a human. Generally, the invention provides polynucleotide, protein, and library production techniques that may be used to produce useful biological constructs, preferably non-immunogenic therapeutic proteins. Exemplary designs include, for example, design of proteins having novel or enhanced characteristics including biochemical and/or biophysical properties. In one embodiment, the methods described herein may be used to develop improved human therapeutics, for example, by designing backbones around active site residues based on human protein fragments in silico to produce variants with desired characteristics such as higher binding affinity, improved stability, better bioavailability, or ease of manufacture while maintaining functionality and no or low immunogenicity. Additionally, the methods described herein may be used to develop combinations of a binding domain, linker and catalytic domain that result in optimal catalytic efficiency. In yet another embodiment, the methods described herein may be used to develop “minimal proteins.” For example, the backbone of the functional area(s) of a protein may be fixed and the chains of this region may be connected with the smallest possible backbone that results in a single, stable molecule. The sequence of the polypeptide may be further optimized to maintain the structure of the backbone. Such minimal proteins may facilitate protein manufacturing and yield proteins with greater stability or higher rates of diffusion. When these proteins are designed using human peptides selected from a library of the invention, these novel proteins are expected to exhibit little or no immunogenicity toward humans.

3. Human Peptide Sequence Libraries and Novel Protein Sequence Libraries

In one aspect, the invention provides a library of sequences of peptide motifs found in human proteins. The library may contain a plurality of sequences of human peptides of a given size range, having more than 4 amino acid residues, preferably more than 5, 6, 7, 8, 9, or 10 amino acid residues, and less than about 50 amino acid residues, preferably less than about 40, 30, 20, or 15 amino acid residues. In one embodiment of the invention, the library comprises sequences of peptides having about 6 to 15 amino acid residues, about 8-12 amino acid residues, or about 8-10 amino acid residues. In certain embodiments, the peptide sequence libraries may contain the sequences of all or substantially all of the peptides from human proteins having a given size range. Human proteins sequences may be obtained from a variety of sources, including, for example, one or more known databases. Suitable known databases include, but not limited to, SCOP (Hubbard, et al., Nucleic Acids Res 27(1):254-256. (1999)); PFAM (Bateman, et al., Nucleic Acids Res 27(1):260-262. (1999)); VAST (Gibrat, et al., Curr Opin Struct Biol 6(3):377-385. (1996)); CATH (Orengo, et al., Structure 5(8):1093-1108. (1997)); PhD Predictor (world wide web at embl-heidelberg.de/predictprotein/predictprotein.html); Prosite (Hofmann, et al., Nucleic Acids Res 27(1):215-219. (1999)); PIR (world wide web at mips.biochem.mpg.de/proj/protseqdb/); GenBank (world wide web at ncbi.nlm.nih.gov/); PDB (world wide web at rcsb.org) and BIND (Bader, et al., Nucleic Acids Res 29(1):242-245. (2001)). Databases providing nucleotide sequences may be used to obtain the corresponding amino acid sequences if desired.

In certain embodiments, the peptide sequence libraries described herein may also include structural information about the peptides. For example, the database may include information about the geometries and conformations of the peptides, such as alpha helix, beta sheet, random coil conformations and disordered regions. In certain embodiments, the peptide sequence libraries may also include additional information regarding the conformation of the peptides such as the relative positions of the alpha carbons of the peptide backbone. The structural information of the peptide member sequences may be derived from actual structures of the peptides, either in an isolated form or in the context of a full length protein. Peptide and protein structural information may be based on X-ray crystallography, solution NMR, fluorescence energy transfer, circular dichroism measurement, or any physical and/or biochemical methodology, or predicted by various available algorithms and models, much refined since the pioneering work of Chow and Fasman (Biochemistry 13, 211-222 (1974)). Examples of secondary structure prediction methods include, but are not limited to, threading (Bryant and Altschul, Curr. Opin. Struct. Biol. 5(2):236-244. (1995)), Profile 3D (Bowie et al., Methods Enzymol. 266(598-616 (1996); MONSSTER (Skolnick et al., J. Mol. Biol. 265(2):217-241. (1997); Rosetta (Simons et al., Proteins 37(S3):171-176 (1999); PSI-BLAST (Altschul and Koonin, Trends Biochem. Sci. 23(11):444-447. (1998)); Impala (Schaffer et al., Bioinformatics 15(12):1000-1011. (1999)); HMMER (McClure et al., Proc. Int. Conf. Intell. Syst. Mol. Biol. 4(155-164 (1996)); Clustal W (world wide web at ebi.ac.uk/clustalw/); BLAST (Altschul et al., J. Mol. Biol. 215(3):403-410. (1990)), helix-coil transition theory (Munoz and Serrano, Biopolymers 41:495, 1997), neural networks, local structure alignment and others (e.g., see in Selbig et al., Bioinformatics 15:1039, 1999). In addition, structures or conformations of peptides and proteins can be predicted based on similar peptides or proteins with known structures using alignment and energy calculation programs.

In another aspect, the invention provides a peptide sequence library having a subset of all human peptides such as the ones described above. For example, the library may comprise sequences of human peptides derived from a particular group of proteins, such as, human bone morphogenic proteins, kinases, phosphatases, cytokines, growth factors, receptors, etc. In another example, the peptide sequence library may contain peptides having a common structure, such as, for example, an alpha helix, beta sheet, random coil or disordered region.

In a preferred embodiment, a peptide sequence library comprises peptides that are produced when human proteins are processed for antigen presentation. For example, the library comprises sequences of peptides that would be produced upon degradation of native proteins by the cellular machinery, e.g., peptides generated by proteasomal cleavage in the cytosol or peptides generated by acid proteases such as asparagine endopeptidases or aspartic proteases (e.g. cathepsin E) in the intracellular vesicles of macrophages, immature dendritic cells, B cells, and other antigen-presenting cells. Cleavage sites of proteins, which form the termini of the resulting peptides when proteins are digested by proteasomes, have been experimentally determined for some proteins, and more recently, have been predicted and peptides from exemplary proteins were experimentally verified, for example, by Khattab et al., Ann. Hematol. 2004 February; 83(2): 107-13. Another tool available for predicting cleavage sites can be found on the Internet at paproc.de (Nussbaum et al., Immunogenetics 2001 March; 53(2):87-94).

In another embodiment, the peptide sequence libraries described herein may contain information about the prevalence of one or more peptides across a population. Such information can be used as a factor to weight selections of peptides from the database as it would be preferable to introduce peptides into a protein scaffold that are found in a high percentage of the world population. These peptides would be recognized as self by a high percentage of the population and therefore be non-immunogenic in such populations. Alternatively, it may be possible to use peptides that are unique to a particular population to design protein therapeutics that are specific to the population. Several versions of the protein therapeutic utilizing different population specific peptides may be produced if the therapeutic protein is to be used for a variety of populations, e.g., a pharmacogenomics type approach to therapeutic protein design. Information about population specific peptides may be obtained, for example, from single nucleotide polymorphism (SNP) databases such as The Single Nucleotide Polymorphism database at NCBI (world wide web at ncbi.nlm.nih.gov/projects/SNP/) and The SNP Consortium, Ltd. at Cold Spring Harbor (world wide web at snp.cshl.org/).

In another aspect, the invention provides methods for designing proteins using the peptide sequence libraries described herein. Using known methods of computational or in silico protein design, a person skilled in the art will be able to carry out de novo protein design, or modify a desired protein, using one or more peptides from a peptide sequence library. For example, a peptide having a structure similar to a portion of a known protein may be selected from the peptide sequence library. Structural similarity between the peptide and a portion of the known protein may be determined by overlaying the three-dimensional peptide structure onto a domain, a motif, or any partial structure of the protein. Such a known protein is herein referred to as a “scaffold protein,” which is more fully described below. Thus, a new protein may be designed by replacing at least one original part of the structure of a scaffold protein with a peptide from the peptide sequence library. One or more parts of the scaffold protein can be replaced. In one embodiment, the scaffold protein is a human protein and one or more portions of the protein may be replaced, for example, to increase structural stability of modify activity of the protein. In another embodiment, the scaffold protein is a non-human protein and all portions of the protein are replaced with peptides from the peptide sequence library. For example, the structure of the scaffold protein may be maintained while replacing the non-human sequences with human peptides that will be recognized as self upon degradation of the protein by the host's immune system. In another embodiment, all possible combinations of two or more peptides from the peptide sequence library can be made in silico, producing a library of newly designed proteins based on the structure of the scaffold protein and the peptide structures of the peptide sequence library. Following the creation of such a library, each hypothetical protein can be computationally evaluated for a property of interest.

The scaffold protein may be any protein, but preferred proteins are those for which a three-dimensional structure is known or can be generated; that is, proteins for which there are three dimensional coordinates for each atom of the protein. Generally this can be determined using X-ray crystallographic techniques, NMR techniques, de novo modeling, homology modeling, etc. In general, if X-ray structures are used, structures at 2 Å resolution or better are preferred, but not required.

Thus, by “scaffold protein” herein is meant a protein for which a library of new proteins is desired. For example, a scaffold protein includes a known protein for which a modified version is desired, for example, a less immunogenic version, a non-immunogenic version, a protein with altered structure, a protein with altered stability, or a protein with altered activity. Alternatively, a scaffold protein may include a de novo protein design that is desired to be produced from the peptide sequence libraries described herein. In certain embodiments, it may be desirable to produce a library of scaffold proteins variants which may be tested for one or more desired characteristics. As will be appreciated by those in the art, any number of scaffold proteins find use in the present invention. Specifically included within the definition of “protein” are fragments and domains of known proteins, including functional domains such as enzymatic domains, binding domains, etc., and smaller fragments, such as turns, loops, etc. That is, portions of proteins may be used as well. In addition, “protein” as used herein includes proteins, oligopeptides and peptides.

The scaffold proteins may be from any organism, including prokaryotes and eukaryotes, with enzymes from bacteria, fungi, extremeophiles such as the archebacteria, insects, fish, animals (particularly mammals and particularly human) and birds all possible. However, as described above, if the scaffold protein is from a non-human source, new proteins preferably are designed by replacing all sequences of the scaffold protein with the sequences found in a peptide sequence library described herein.

Suitable scaffold proteins include, but are not limited to, pharmaceutical proteins, including ligands, cell surface receptors, antigens, antibodies, cytokines, hormones, transcription factors, signaling modules, cytoskeletal proteins and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phosphatases. Suitable enzymes are listed in the Swiss-Prot enzyme database. Suitable protein backbones include, but are not limited to, all of those found in the protein database compiled and serviced by the Research Collaboratory for Structural Bioinformatics (RCSB, formerly the Brookhaven National Lab; see world wide web at rcsb.org).

Specifically, preferred scaffold proteins include, but are not limited to, those with known structures (including variants) including cytokines (IL-1ra (+receptor complex), IL-1 (receptor alone), IL-1a, IL-1b (including variants and or receptor complex), IL-2, IL-3, IL-4, IL-5, IL-6, IL-8, IL-10, IFN-β, INF-γ, IFN-α-2a; IFN-α-2B, TNF-α; CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration inhibitory Factor, Human Glycosylation-Inhibiting Factor, Human Rantes, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve growth factor, Brain Derived Neurotrophic Factor, Ciliary Neurotrophic Factor, Platelet Derived Growth Factor, Human Hepatocyte Growth Factor, Glial Cell-Derived Neurotrophic Factor, (as well as the at least 55 cytokines in PDB)); Erythropoietin; other extracellular signaling moeities, including, but not limited to, hedgehog Sonic, hedgehog Desert, hedgehog Indian, hCG; coagulation factors including, but not limited to, TPA and Factor VIIa; transcription factors, including but not limited to, p53, p53 tetramerization domain, Zn fingers (of which more than 12 have structures), homeodomains (of which 8 have structures), leucine zippers (of which 4 have structures); antibodies, including, but not limited to, cFv; viral proteins, including, but not limited to, hemagglutinin trimerization domain and HIV Gp41 ectodomain (fusion domain); intracellular signaling modules, including, but not limited to, SH2 domains (of which 8 structures are known), SH3 domains (of which 11 have structures), and Pleckstin Homology Domains; receptors, including, but not limited to, the extracellular region of human tissue factor cytokine-binding region of Gp 130, G-CSF receptor, erythropoietin receptor, Fibroblast Growth Factor receptor, TNF receptor, IL-1 receptor, IL-1 receptor/IL1ra complex, IL-4 receptor, INF-γ receptor alpha chain, MHC Class I, MHC Class II, T Cell Receptor, Insulin receptor, insulin receptor tyrosine kinase and human growth hormone receptor.

Once a scaffold protein is selected, a protein sequence library is created by computational processing, substituting parts of the scaffold protein sequence with members of the peptide sequence library, thus creating an immunologically human protein which retains the structure of the scaffold protein. Generally speaking, in some embodiments, the goal of the computational processing is to determine a set of optimized protein sequences, typically using known or to be developed computational processing techniques. By “optimized protein sequence” herein is meant a sequence that best fits the mathematical equations of the computational process. As will be appreciated by those in the art, a global optimized sequence is the one sequence that best fits the equations (for example, when protein design automation (PDA) is used, the global optimized sequence is the sequence that best fits Equation 1, below); i.e. the sequence that has the lowest energy of any possible sequence. However, there are any number of sequences that are not the global minimum but that have low energies.

In a preferred embodiment, using a publicly available program, a human peptide sequence library of the invention is screened for peptides that structurally align with parts of the scaffold protein. Identified peptides may then be used to replace those parts of the scaffold protein with which they structurally align. There are a wide variety of such structural alignment programs known. See, for example, VAST from the NCBI (world wide web at ncbi.nlm.nih.gov: 80/StructureNAST/vast.shtml); SSAP (Orengo and Taylor, Methods Enzymol 266(617-635 (1996)) SARF2 (Alexandrov, Protein Eng 9(9):727-732. (1996)) CE (Shindyalov and Bourne, Protein Eng 11(9):739-747. (1998)); (Orengo et al., Structure 5(8):1093-108 (1997); Dali (Holm et al., Nucleic Acid Res. 26(1):316-9 (1998), all of which are incorporated by reference. When replacing a disordered region of a scaffold protein (e.g., a region that has no defined structure), preference may be given to replacement peptides that have (or are predicted to have) no known structure as well (e.g., disordered).

The libraries can be generated in a variety of ways. In essence, any method that can result in either the relative ranking of the possible sequences of a protein based on measurable stability parameters, or a list of suitable sequences can be used. As will be appreciated by those skilled in the art, any of the methods described herein or known in the art may be used alone, or in combination with other methods. In a preferred embodiment, the computational method used to generate the protein library is Protein Design Automation (PDA), as is described in U.S. Pat. No. 6,269,312 and PCT Publication No. WO 98/47089, which are expressly incorporated herein by reference. PDA, viewed broadly, has three components that may be varied to alter the output (e.g., the library): the scoring functions used in the process; the filtering technique, and the sampling technique.

Briefly, PDA can be described as follows. A known protein structure is used as the starting point. The residues to be optimized are then identified, which may be the entire sequence or subset(s) thereof. The side chains of any positions to be varied are then removed. The resulting structure consisting of the protein backbone and the remaining side chains is called the template. Each variable residue position is then preferably classified as a core residue, a surface residue, or a boundary residue; each classification defines a subset of possible amino acid residues for the position (for example, core residues generally will be selected from the set of hydrophobic residues, surface residues generally will be selected from the hydrophilic residues, and boundary residues may be either). Each amino acid can be represented by a discrete set of all allowed conformers of each side chain, called rotamers. Thus, to arrive at an optimal sequence for a backbone, all possible sequences of rotamers must be screened, where each backbone position can be occupied either by each amino acid in all its possible rotameric states, or a subset of amino acids, and thus a subset of rotamers.

Two sets of interactions are then calculated for each rotamer at every position: the interaction of the rotamer side chain with all or part of the backbone (the “singles” energy, also called the rotamer/template or rotamer/backbone energy), and the interaction of the rotamer side chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a “Dead End Elimination” (DEE) calculation is performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence which represents the global optimum energy.

Once the global solution has been found, a Monte Carlo search may be done to generate a rank-ordered list of sequences in the neighborhood of the DEE solution. Starting at the DEE solution, random positions are changed to other rotamers, and the new sequence energy is calculated. If the new sequence meets the criteria for acceptance, it is used as a starting point for another jump. After a predetermined number of jumps, a rank-ordered list of sequences is generated. Monte Carlo searching is a sampling technique to explore sequence space around the global minimum or to find new local minima distant in sequence space. As is further outlined below, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Similarly, for all the sampling techniques, the acceptance criteria of whether a sampling jump is accepted can be altered.

In practice, as outlined in U.S. Pat. No. 6,269,312, the protein backbone (comprising (for a naturally occurring protein) the nitrogen, the carbonyl carbon, the α-carbon, and the carbonyl oxygen, along with the direction of the vector from the α-carbon to the β-carbon) may be altered prior to the computational analysis, by varying a set of parameters called supersecondary structure parameters. Once a protein structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths. In a preferred embodiment, this is done by conducting a number of steps of conjugate gradient minimization (Mayo et al., J. Phys. Chem. 94:8897 (1990)) of atomic coordinate positions to minimize the Dreiding force field with no electrostatics. Generally from about 10 to about 250 steps is preferred, with about 50 being most preferred.

The protein backbone structure contains at least one variable residue position. As is known in the art, the residues, or amino acids, of proteins are generally sequentially numbered starting with the N-terminus of the protein. Thus a protein having a methionine at its N-terminus is said to have a methionine at residue or amino acid position 1, with the next residues as 2, 3, 4, etc. At each position, the wild type (i.e. naturally occurring) protein may have one of at least 20 amino acids, in any number of rotamers. By “variable residue position” herein is meant an amino acid position of the protein to be designed that is not fixed in the design method as a specific residue or rotamer, generally the wild-type residue or rotamer.

In a preferred embodiment, all of the residue positions of the protein are variable. That is, every amino acid side chain may be altered in the methods of the present invention. This is particularly desirable for smaller proteins, although the present methods allow the design of larger proteins as well. While there is no theoretical limit to the length of the protein which may be designed this way, there may is a practical computational limit.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid. Alternatively, the methods of the present invention may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues. Thus, for example, it may be possible to change only a few (or one) residues, or most of the residues, with all possibilities in between.

In a preferred embodiment, residues which can be fixed include, but are not limited to, structurally or biologically functional residues; alternatively, biologically functional residues may specifically not be fixed. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

In a preferred embodiment, each variable position is classified as either a core, surface or boundary residue position, although in some cases, as explained below, the variable position may be set to glycine to minimize backbone strain. In addition, as outlined herein, residues need not be classified, they can be chosen as variable and any set of amino acids may be used. Any combination of core, surface and boundary positions can be utilized: core, surface and boundary residues; core and surface residues; core and boundary residues, and surface and boundary residues, as well as core residues alone, surface residues alone, or boundary residues alone.

The classification of residue positions as core, surface or boundary may be done in several ways, as will be appreciated by those in the art. In a preferred embodiment, the classification is done via a visual scan of the original protein backbone structure, including the side chains, and assigning a classification based on a subjective evaluation of one skilled in the art of protein modeling. Alternatively, a preferred embodiment utilizes an assessment of the orientation of the Cα-Cβ vectors relative to a solvent accessible surface computed using only the template Cα atoms, as outlined in U.S. Pat. No. 6,269,312 and PCT Publication No. WO 98/47089. Alternatively, a surface area calculation can be done.

Once each variable position is classified as either core, surface or boundary, a set of amino acid side chains, and thus a set of rotamers, is assigned to each position. That is, the set of possible amino acid side chains that the program will allow to be considered at any particular position is chosen. The choice of amino acid side chains may be based, for example, on the sequences of peptides in the peptide sequence library, and/or based on the similarity of local secondary or tertiary structure of the protein to the structure of peptides in the peptide sequence library, as determined by a structure alignment program. Subsequently, once the possible amino acid side chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the a scaling factor of the van der Waals scoring function, described below, is low, methionine is removed from the set), and the rotamer set for each core position potentially includes rotamers for these eight amino acid side chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be). Additionally, in some preferred embodiments, a set of 18 naturally occurring amino acids (all except cysteine and proline, which are known to be particularly disruptive) are used.

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins which dimerize or multimerize, or have ligand binding sites, may contain hydrophobic surface residues, etc. In addition, residues that do not allow helix “capping” or the favorable interaction with an a-helix dipole may be subtracted from a set of allowed residues. This modification of amino acid groups is done on a residue by residue basis.

In a preferred embodiment, proline, cysteine and glycine are not included in the list of possible amino acid side chains, and thus the rotamers for these side chains are not used. However, in a preferred embodiment, when the variable residue position has a φ angle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once the group of potential rotamers is assigned for each variable residue position, processing proceeds as outlined in U.S. Pat. No. 6,269,312 and PCT Publication No. WO 98/47089. This processing step entails analyzing interactions of the rotamers with each other and with the protein backbone to generate optimized protein sequences. Simplistically, the processing initially comprises the use of a number of scoring functions to calculate energies of interactions of the rotamers, either to the backbone itself or other rotamers. Preferred PDA scoring functions include, but are not limited to, a Van der Waals potential scoring function, a hydrogen bond potential scoring function, an atomic solvation scoring function, a secondary structure propensity scoring function and an electrostatic scoring function. As is further described below, at least one scoring function is used to score each position, although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1:
Eₜₒₜₐₗ=nE_{vdw}+nEₐₛ+nEₕ−bonding+nEₛₛ+nE_{elec} Equation 1

In Equation 1, the total energy is the sum of the energy of the van der Waals potential (E_{vdw}), the energy of atomic solvation (Eₐₛ), the energy of hydrogen bonding (Eₕ-bonding), the energy of secondary structure (Eₛₛ) and the energy of electrostatic interaction (E_{elec}). The term n is either 0 or 1, depending on whether the term is to be considered for the particular residue position.

As outlined in U.S. Pat. No. 6,269,312 and PCT Publication No. WO 98/47089, any combination of these scoring functions, either alone or in combination, may be used. Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered. The term “portion”, as used herein, with regard to a protein refers to a fragment of that protein. This fragment may range in size from 5, 6, 7, 8, 9, 10, 12, 15, or more, amino acid residues to the entire amino acid sequence minus one amino acid. Accordingly, the term “portion”, as used herein, with regard to a nucleic refers to a fragment of that nucleic acid. This fragment may range in size from 10 nucleotides to the entire nucleic acid sequence minus one nucleotide.

In a preferred embodiment, the first step of the computational processing is done by calculating two sets of interactions for each rotamer at every position: the interaction of the rotamer side chain with the template or backbone (the “singles” energy), and the interaction of the rotamer side chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the E_{HB} is calculated for each possible rotamer at every variable position. Similarly, for the van der Waals scoring function, every atom of the rotamer is compared to every atom of the template (generally excluding the backbone atoms of its own residue), and the E_{vdw} is calculated for each possible rotamer at every variable residue position. In addition, generally no van der Waals energy is calculated if the atoms are connected by three bonds or less. For the atomic salvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the lower the energy.

For the calculation of “doubles” energy (rotamer/rotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{HB} is calculated for each possible rotamer pair for any two variable positions. Similarly, for the van der Waals scoring function, every atom of the first rotamer is compared to every atom of every possible second rotamer, and the E_{vdw} is calculated for each possible rotamer pair at every two variable residue positions. For the atomic solvation scoring function, the surface of the first rotamer is measured against the surface of every possible second rotamer, and the Eₐₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the lower the energy.

In addition, as will be appreciated by those in the art, a variety of force fields that can be used in the PDA calculations can be used, including, but not limited to, Dreiding I and Dreiding II (Mayo et al., J. Phys. Chem. 94:8897 (1990)), AMBER 1.1 and 3.0 (Weiner et al., J. Amer. Chem. Soc. 106:765 (1984); Weiner et al., J. Comp. Chem. 7:230 (1986), and Singh et al., Proc. Natl. Acad. Sci. USA 82:755-759); MM2 and MM3 (Allinger, J. Chem. Soc. 99:8127 (1977) and Allinger et al., J. Amer. Chem. Soc. 111:8551 (1989), Liljefors et al., J. Comp. Chem. 8:1051 (1987)); MMP2 (Sprague et al., J. Comp. Chem. 8:581 (1987)); CHARMM and CHARMM22 (Brooks et al., J. Comp. Chem. 4:187 (1983)); GROMOS (Scott et al., J. Phys. Chem., 103: 3596 (1999)); OPLS and OPLS-M (Jorgensen et al., J. Am. Chem. Soc., 110: 1657ff (1988); Jorgensen et al., J. Am. Chem. Soc., 112:4768ff (1990) and Jorgensen et al., J. Am. Chem. Soc., 118:11225 (1996)); BOSS Ver. 4.1 (Jorgensen, Yale University: New Haven, Conn. (1999)); UNRES ((United Residue Forcefield) Liwo et al., Protein Science, 2:1697 (1993); Liwo et al., Protein Science 2:1715 (1993); Liwo et al., J. Comp. Chem. 18:849 (1997); Liwo et al., J. Comp. Chem., 18:874 (1997); Liwo et al., J. Comp. Chem., 19: 259 (1998)); Forcefield for Protein Structure Prediction (Liwo et al., Proc. Natl. Acad. Sci. USA 96:5482 (1999)); ECEPP/3 (Liwo et al., J. Protein Chem. 13(4):375 (1994)); cvff3.0 (Dauber-Osguthorpe et al., Proteins: Structure, Function and Genetics, 4: 31 (1988)); cff91 (Maple et al., J. Comp. Chem. 15: 162 (1994)). Note that the DISCOVER (cvff and cff91) and AMBER forcefields are used in the INSIGHT molecular modeling package (Biosym/MSI, San Diego, Calif.) and CHARMM is used in the QUANTA molecular modeling package (Biosym/MSI, San Diego, Calif.), all of the programs and algorithms cited in this paragraph are expressly incorporated by reference. In addition, there are computational methods based on forcefield calculations such as the self-consistent mean-field (SCMF) method that can be used as well, see Delarue et al. Proc. Pacific Symp. Biocomput. 109-21 (1997), Koehl et al., J. Mol. Biol. 239:249 (1994); Koehl et al., Nat. Struc. Biol. 2:163 (1995); Koehl et al., Curr. Opin. Struct. Biol. 6:222 (1996); Koehl et al., J. Mol. Biol. 293:1183 (1999); Koehl et al., J. Mol. Biol. 293:1161 (1999); Lee, J. Mol. Biol. 236:918 (1994); and Vasquez, Biopolymers 36:53 (1995); all of which are expressly incorporated by reference.

Once the singles and doubles energies are calculated and stored, the next step of the computational processing may occur. As outlined in U.S. Pat. No. 6,269,312 and PCT Publication No. WO 98/47089, preferred embodiments utilize a Dead End Elimination (DEE) step, and preferably a Monte Carlo step. In a preferred embodiment, a variety of filtering techniques can be done, including, but not limited to, DEE and its related counterparts. Additional filtering techniques include, but are not limited to branch-and-bound techniques for finding optimal sequences (Gordon and Majo, Structure Fold. Des. 7:1089-98, 1999), and exhaustive enumeration of sequences. It should be noted however, that some techniques may also be done without any filtering techniques; for example, sampling techniques can be used to find good sequences, in the absence of filtering.

As will be appreciated by those in the art, once an optimized sequence or set of sequences is generated, (or again, these need not be optimized or ordered) a variety of sequence space sampling methods can be done, either in addition to the preferred Monte Carlo methods, or instead of a Monte Carlo search. That is, once a sequence or set of sequences is generated, preferred methods utilize sampling techniques to allow the generation of additional, related sequences for testing.

These sampling methods can include the use of amino acid substitutions, insertions or deletions, or recombinations of one or more sequences. As outlined herein, a preferred embodiment utilizes a Monte Carlo search, which is a series of biased, systematic, or random jumps. However, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Jumps where multiple residue positions are coupled (two residues always change together, or never change together), jumps where whole sets of residues change to other sequences (e.g., recombination). Similarly, for all the sampling techniques, the acceptance criteria of whether a sampling jump is accepted can be altered, to allow broad searches at high temperature and narrow searches close to local optima at low temperatures. See Metropolis et al., J. Chem Phys v21, pp 1087, 1953, hereby expressly incorporated by reference.

In a preferred embodiment, the scoring functions may be altered, for example, the scoring functions outlined above may be biased or weighted in a variety of ways. For example, a bias towards or away from a reference sequence or family of sequences can be used; for example, a bias towards wild-type or homolog residues may be used. Similarly, the entire protein or a fragment of it may be biased; for example, the active site may be biased towards wild-type residues, or domain residues biased towards a particular desired physical property. Furthermore, a bias towards or against increased energy can be generated. Additional scoring function biases include, but are not limited to applying electrostatic potential gradients or hydrophobicity gradients, adding a substrate or binding partner to the calculation, or biasing towards a desired charge or hydrophobicity.

In addition, in an alternative embodiment, there are a variety of additional scoring functions that may be used. Additional scoring functions include, but are not limited to torsional potentials, residue pair potentials, or residue entropy potentials. Such additional scoring functions can be used alone, or as functions for processing the library after it is scored initially. For example, a variety of functions derived from data on binding of peptides to MHC (Major Histocompatibility Complex) can be used to rescore a library in order to eliminate proteins containing sequences which can potentially bind to MHC, i.e. potentially immunogenic sequences, further lowering the likelihood of immunogenicity.

In certain embodiments, the methods described herein for designing therapeutic proteins may also take into consideration the junctions that are produced upon introduction of a peptide into a protein scaffold. Preferably, the in silico step will allow selection of a composite protein wherein the peptides, protein scaffold and new junctions formed between the inserted peptide and protein scaffold all look human, e.g., would be recognized as self by a host organism. For example, using A as the sequence of the original protein scaffold and B as the sequence of the peptide insert, a new sequence AAAAAAAAABBBBBBAAAAAAAAA would be created upon introduction of the peptide into the protein scaffold. In this example, the A sequence and the B sequence would have been selected for non-immunogenicity (or low immunogenicity) in a desired host. However, it is also desirable to test the junctions formed between the A and B sequences for non-immunogenicity (or low immunogenicity). This may be conducted by selecting a window equivalent to an antigenic fragment, for example, a T-cell epitope, antibody epitope, etc. Exemplary windows may be, for example, from about 6 to about 15 amino acids in length, about 8 to 12 amino acids in length, about 8 to 10 amino acids in length, or about 7, 8, 9, 10, 11, or 12 amino acids in length. In the example given above, if a window of 6 amino acids is selected, then all junction sequences of 6 amino acids may be tested for immunogenicity, e.g., AAAAAB, AAAABB, AAABBB, AABBBB, ABBBBB, BBBBBA, BBBBAA, BBBAAA, BBAAAA, and BAAAAA. These junction sequences may be compared to a database of human peptides as described herein to ensure that none of the sequences will produce an undesirable immunogenic response in the desired host. Alternatively, such sequences may be run through an algorithm to predict the immunogenicity of such sequences. Methods for predicting the potential immunogenicity of a peptide are known in the art (see e.g., T. Sturniolo et al., Nature Biotech. 17: 555-561 (1999)) or are available through commercial sources (see e.g., world wide web at antitope.co.uk, algonomics.com and epivax.com). Such methods may be used to select combinations of protein scaffolds and peptides that will look fully human, e.g., all possible peptides of a certain length will be recognized as self. To achieve such a fully human peptide, it may be necessary to alter the sequence of the peptide being inserted into the protein scaffold or alter the location in the protein scaffold into which the peptide is being inserted to ensure that the junction sequences will be non-immunogenic. In certain embodiments, it may not be possible to find a desired peptide sequence and location within the scaffold such that all peptide sequences are non-immunogenic. In such situations, the predicted immunogenicity of the junction peptides may be selected such that they have the lowest possible immunogenicity among the available possibilities. The methods for selecting non-immunogenic (or low immunogenicity) junction peptides described above are equally applicable to the situation where proteins are being designed de novo from a number of peptide segments.

In other embodiments, the immunogenicity of junctions may be considered in combination with protease cleavage sites. For example, a peptide may be inserted into a scaffold protein such that the junctions between the peptide and scaffold protein correspond to protease cleavage sites. As such, even if the junctions themselves would yield immunogenic peptides, the placement of the peptide between protease cleavage sites will ensure that none of the junction peptides will be generated, e.g., the designed protein will be predictably cleaved into peptides derived from either the scaffold protein or inserted peptide that have already been assessed for their immunogenicity and no junction peptides will be created.

In addition, as outlined above, other computational methods useful for the practice of the present invention are known, including, but not limited to, sequence profiling (Bowie and Eisenberg, Science 253: 164 (1991)), rotamer library selections (Dahiyat and Mayo, Protein Sci. 5: 895 (1996); Dahiyat and Mayo, Science 278: 82 (1997); Desjarlais and Handel, Protein Sci. 4: 2006 (1995); Harbury et al., Proc. Nat. Acad. Sci. USA 92: 8408 (1995); Kono et al., Proteins: Structure, Function and Genetics 19: 244-255 (1994); Hellinga and Richards, Proc. Nat. Acad. Sci. USA 91: 5803 (1994)); and residue pair potentials (Jones, Protein Sci. 3: 567 (1994); PROSA (Heindlich et al., J. Mol. Biol. 216:167 (1990); THREADER (Jones et al., Nature 358:86 (1992), and other inverse folding methods such as those described by Simons et al. (Proteins, 34:535 (1999)), Levitt and Gerstein (Proc. Nat. Acad. Sci. USA, 95:5913 (1998)), Godzik and Skolnick, Proc. Nat. Acad. Sci. USA, 89: 12098; Godzik et al. (J. Mol. Biol. 227:227 (1992)) and two profile methods (Gribskov et al., Proc. Nat. Acad. Sci. USA 84:4355 (1987) and Fischer and Eisenberg, Protein Sci. 5:947 (1996), Rice and Eisenberg, J. Mol. Biol. 267:1026 (1997)), all of which are expressly incorporated by reference. In addition, other computational methods such as those described by Koehl and Levitt (J. Mol. Biol. 293:1161 (1999); J. Mol. Biol. 293:1183 (1999); expressly incorporated by reference) can be used to create a protein sequence library for improved properties and function.

3. Biosynthetic Libraries

In another aspect, the invention provides biosynthetic libraries comprising a plurality of synthetic DNAs of known and planned, as opposed to randomized, sequence. For example, a biosynthetic nucleic acid library may comprise polynucleotides encoding the peptides of a peptide sequence library as described above. The peptides of the library can be selected or screened for species having a predetermined property or set of properties, including functional or structural properties. The polynucleotides forming the library preferably are chemically synthesized. In an exemplary embodiment, the library polynucleotides are assembled from chemically synthesized oligonucleotides using techniques such as those set forth herein. The library polynucleotides may have reading frames that exploit consistent codon usage patterns so as to promote similar expression levels in a selected cellular or cell free expression system, e.g., a ribosomal expression system, a phage expression system, or an E. coli expression system. Preferably, the oligonucleotides are synthesized in parallel. It is also preferred to assemble the genes in parallel from the chemically synthesized oligonucleotides.

Libraries described herein may be produced by a variety of methods available to one of skill in the art as described herein and in U.S. Ser. No. 60/643,813, filed Jan. 13, 2005, the disclosure in which is incorporated by reference herein in its entirety, which permit relatively inexpensive, rapid, and high fidelity construction of essentially any polynucleotide desired. Thus, in one embodiment, polynucleotides suitable for construction of a polynucleotide library may be produced, for example, using a nucleic acid array for the direct fabrication of DNA or other nucleic acid molecules of any desired sequence and of indefinite length. Sections or segments of the desired nucleic acid molecule are fabricated on an array, such as by way of a parallel nucleic acid synthesis process using an array synthesizer instrument. After the synthesis of the segments, the segments are assembled to make the desired molecule. In essence the technique permits the quick easy and direct synthesis of nucleic acid molecule for any purpose in a simple and quick synthesis process.

For example, in one embodiment, libraries may be constructed by hybridization based oligonucleotide assembly of overlapping complementary oligonucleotides (see e.g., Zhou et al., Nucleic Acids Res., 32: 5409-5417 (2004); Richmond et al., Nucleic Acids Res. 32: 5011-5018 (2004); Tian et al. Nature 432: 1050-1054 (2004); and Carr et al. Nucleic Acids Res. 32: e162 (2004)). For example, oligonucleotides having complementary, overlapping sequences may be synthesized on a chip and then eluted off. The oligonucleotides then self assemble based on hybridization of the complementary regions. This technique permits the production of long molecules of DNA having high fidelity.

One salient feature of this technique relates to permissible use of low-purity arrays, e.g., arrays having features of less than 10 percent purity with respect to any given nucleic acid sequence. The utility of the low-purity arrays arises from the ability to correct errors occurring in the assembled constructs.

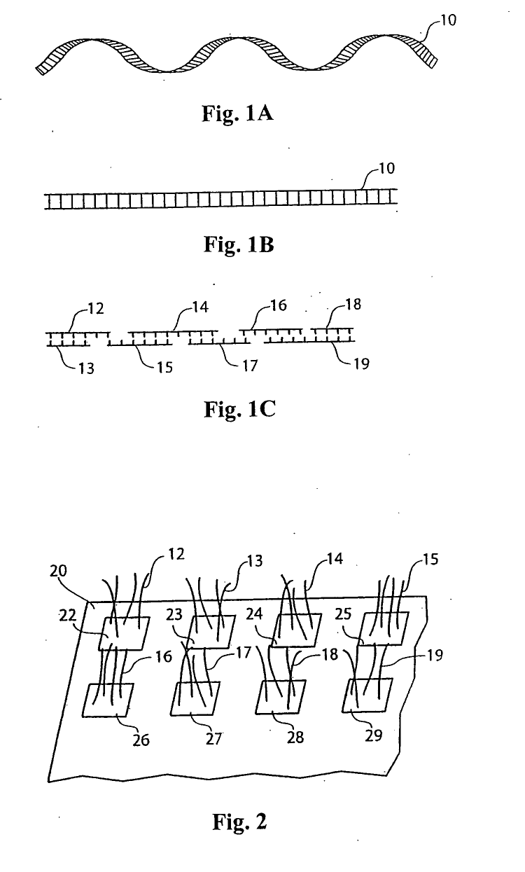

An illustration of the direct fabrication of a relatively simple DNA molecule is described in the figures. In FIG. 1, at 10, a double stranded DNA molecule of known sequence is illustrated. That same molecule is illustrated in both the familiar double helix shape in FIG. 1A, as well as in an untwisted double stranded linear shape shown in FIG. 1B. Assume, for purposes of this illustration, that the DNA molecule is broken up into a series of overlapping single smaller stranded DNA molecule segments, indicated by the reference numerals 12 through 19 in FIG. 1C. The even numbered segments are on one strand of the DNA molecule, while the odd numbered segments form the opposing complementary strand of the DNA molecule. The single stranded molecule segments can be of any reasonable length, but can be conveniently all of the same length which, for purposes of this example, might be 100 base pairs in length. Since the sequence of the molecule 10 of FIG. 1A is known, the sequence of the smaller DNA segments 12 through 19 can be defined simply be breaking the larger sequence into overlapping sequences each of, e.g., 75 to 100 base pairs. In the normal nomenclature of the art, the DNA sequences on the microarray are sometimes referred to as probes because of the intended use of the DNA sequences to probe biological samples. Here these same sequences are referred to as DNA segments, also because of the intended use of these sequences.

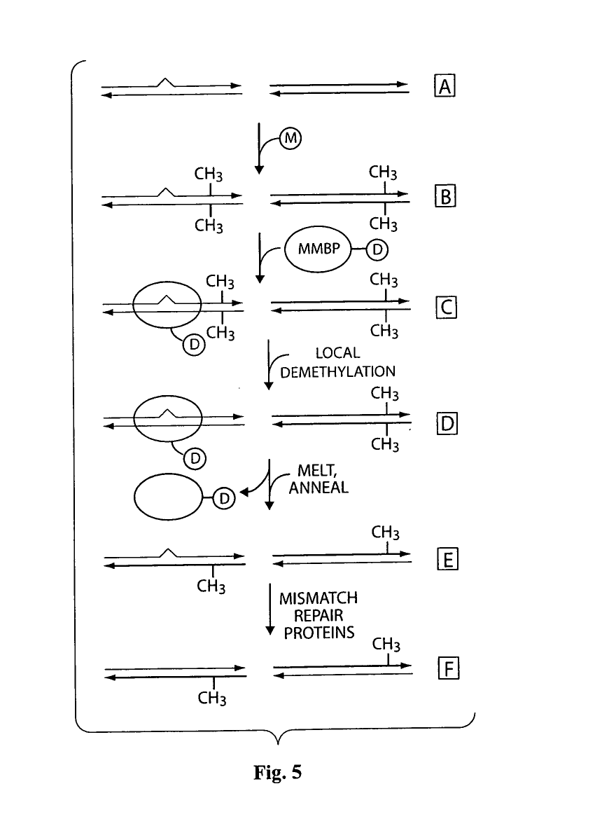

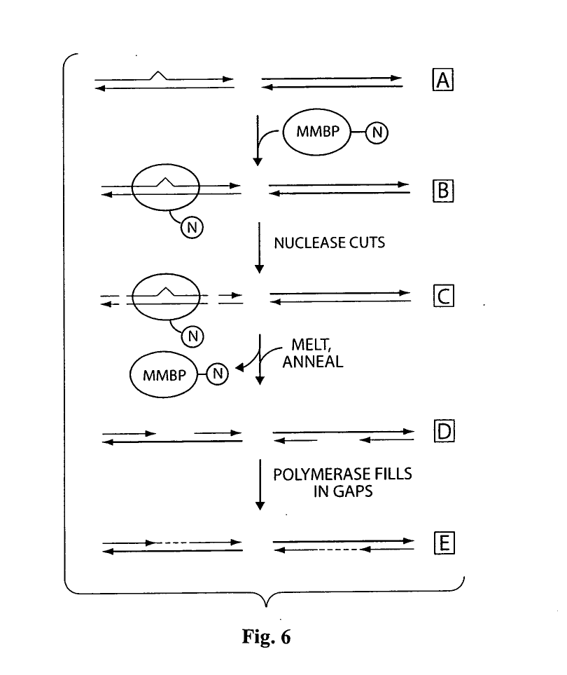

The information about the sequence of the segments 12-19 is then used to construct a new totally fabricated DNA molecule. This process is initiated by constructing a microarray of single stranded DNA segments on a common substrate. This process is illustrated in FIG. 2. Each of the single stranded segments 12 through 19 is constructed in a single cell, or feature, of a DNA microarray indicated at 20. Each of the DNA segments is fabricated in situ in a corresponding feature indicated by reference numbers 22 through 29. Such a microarray is preferably constructed using a maskless array synthesizer (MAS), as for example of the type described in published PCT Publication No. WO 99/42813 and in corresponding U.S. Pat. No. 6,375,903, the disclosure of each of which is herein incorporated by reference. Other examples are known of maskless instruments which can fabricate a custom DNA microarray in which each of the features in the array has a single stranded DNA molecule of desired sequence. The preferred type of instrument is the type shown in FIG. 5 of U.S. Pat. No. 6,375,903, based on the use of reflective optics. It is a desirable that this type of maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no difference to the process.

The MAS instrument may be used in the form it would normally be used to make microarrays for hybridization experiments, but it may also comprise features specifically adapted for the compositions, methods and systems described herein. For example, it may be desirable to substitute a coherent light source, i.e. a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization.

Once the fabrication of the DNA microarray is completed, the single stranded DNA molecule segments on the microarray are then freed or eluted from the substrate on which they were constructed. The particular method used to free the single stranded DNA segments is not critical, several techniques being possible. The DNA segment detachment method most preferred is a method which will be referred to here as the safety-catch method. Under the safety-catch approach, the initial starting material for the DNA strand construction in the microarray is attached to the substrate using a linker that is stable under the conditions required for DNA strand synthesis in the MAS instrument conditions, but which can be rendered labile by appropriate chemical treatment. After array synthesis, the linker is first rendered labile and then cleaved to release the single stranded DNA segments. The preferred method of detachment for this approach is cleavage by light degradation of a photo-labile attachment group.

The single stranded DNA molecules are suspended in a solution under conditions which favor the hybridization of single stranded DNA strands into double stranded DNA. Under these conditions, the single stranded DNA segments will automatically begin to assemble the desired larger complete DNA sequence. This occurs because, for example, the 3′ half of the DNA segment 12 will either preferentially or exclusively hybridize to the complementary half of the DNA segment 13. This is because of the complementary nature of the sequences on the 3′ half of the segment 12 and the sequence on the 5′ half of the segment 13. The half of the segment 13 that did not hybridize to the segment 12 will then, in turn, hybridize to the 3′ half of the segment 14. This process will continue spontaneously for all of the segments freed from the microarray substrate. By this process, a DNA assembly similar to that indicated in FIG. 1C is created. By joining the aligned single stranded DNA molecules to each other, as can be done with a DNA ligase, the DNA molecule 10 of FIG. 1A is completed. The number of copies of the molecule created will be proportional to the number of identical segments synthesized in each of the features in the microarray 20. It may also be desirable to assist the assembly of the completed DNA molecule be performing one of a number of types of sub-assembly reactions. Several alternatives for such reactions are described below.

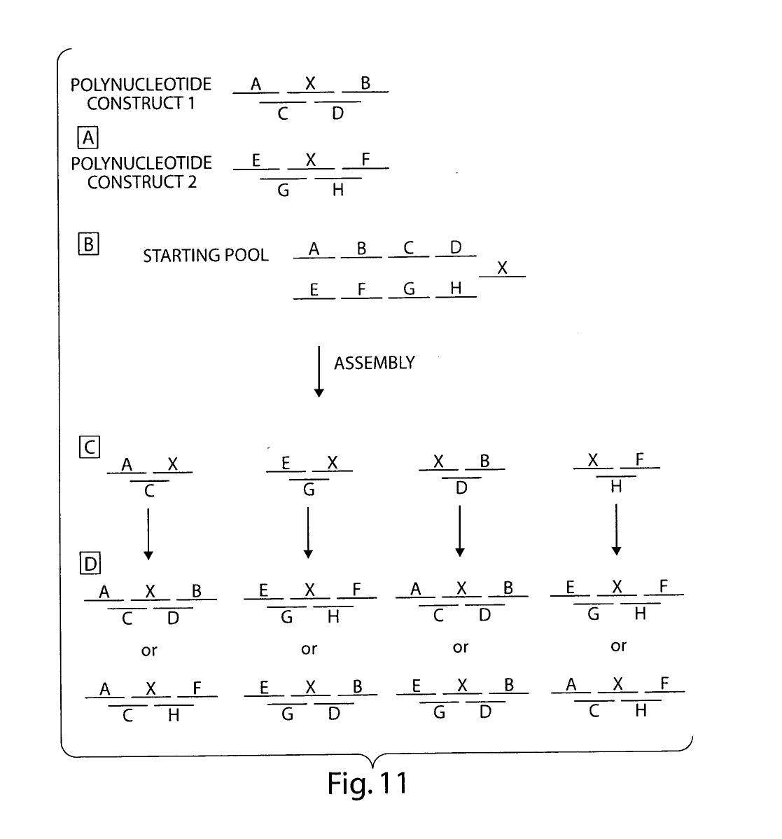

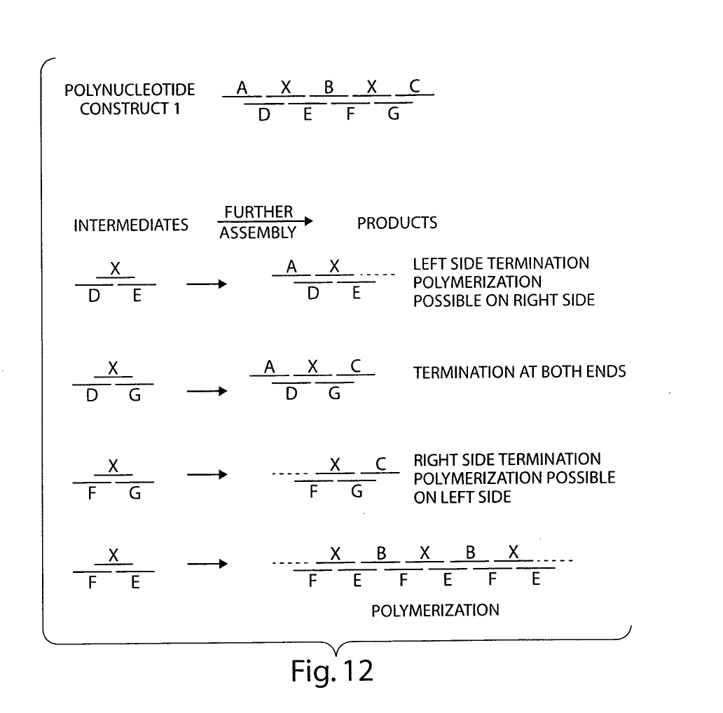

When conducting polymerase assembly multiplexing (PAM), homologous oligonucleotides can potentially act as crossover points leading to a mixture of full length products (FIGS. 11 and 12). Depending on the application, this can be a useful source of diversity, or a complication necessitating an additional separation step to obtain only the desired products. We have now discovered two strategies for accomplishing the selective separation of desired sequences from a mixture of crossover products: (1) selection by intermediate circularization and (2) selection by size. Both apply to PAM of polynucleotide constructs with one or more internal homologous regions.

In PAM (Tian et al., Nature 432: 1050-1054 (2004)), the order in which the oligonucleotide starting materials assemble to form polynucleotide constructs is defined by the mutual 5′ and 3′ complementarities of the oligonucleotides (Mullis et al., Cold Spring Harb. Symp. Quant. Biol. 51 pt 1: 263-273). The ends of each oligonucleotide can anneal to exactly one other oligonucleotide (except for the oligonucleotides at the end of a finished gene, which have a free end). This specificity of annealing ensures that only the desired full-length gene sequences will be assembled.

If there are sufficiently long regions of high homology among the genes to be synthesized in multiplexed format, however, this specificity can be lost. For example, when trying to synthesize two or more polynucleotide constructs that contain a highly homologous (or even identical) region X in a single pool, the common homologous region could lead to various assembled products in addition to the polynucleotide constructs of interest (see FIG. 11). This situation may arise when the homologous region X is at least as long as the construction oligonucleotide. This may occur, for example, when synthesizing polynucleotide constructs that encode closely related protein variants or proteins that share common domains. For example, as shown in FIG. 11, A, B, C, D, E, F, G, H and X denote non-homologous construction oligonucleotides. By design, the 5′ end of X can hybridize with both C and G, and the 3′ end of X can hybridize with both D and H. This does not present a complication if the two sets of oligonucleotides do not come into contact with each other (e.g., they are in separate pools). However, if synthesis is performed in a single well, 4 distinct full-length products will be formed (identified by top strand only): AXB, AXF, EXB, and EXF (see FIG. 11D). Therefore, when dealing with a homologous region, the number of different products that may be formed is sx+1, where s is the number of homologous sequences and x is the number of internal crossover points.

Internal homologous regions (e.g., two regions contained in the same sequence which are highly homologous or identical) are a special case because they have the potential to lead to polymerization in PAM. As shown in FIG. 12, assembly of the AXBXC nucleic acid (represented by the top strand only) could lead to a family of products represented by AX(BX)nC, where n is any nonnegative integer. The number of products generated by this assembly is theoretically infinite.

In certain embodiments, it may be desirable to allow this type of combinatorial complexity to occur. For example, this crossover feature of PAM can be exploited to quickly and cheaply generate large combinatorial libraries for applications such as domain shuffling for protein design, or creation of a library of proteins from a peptide sequence library as described herein, etc.

In other embodiments, it is desirable to minimize or eliminate combination complexity and synthesize only a defined set of homologous sequences. This may be achieved by separately synthesizing genes containing homologous regions (to prevent crossover), for example, using separate pools that are mixed together in an ordered fashion to prevent crossover products. Alternatively, a variety of genes with homologous regions may be synthesized in a single pool and the undesired products may be removed using the separation techniques described below.

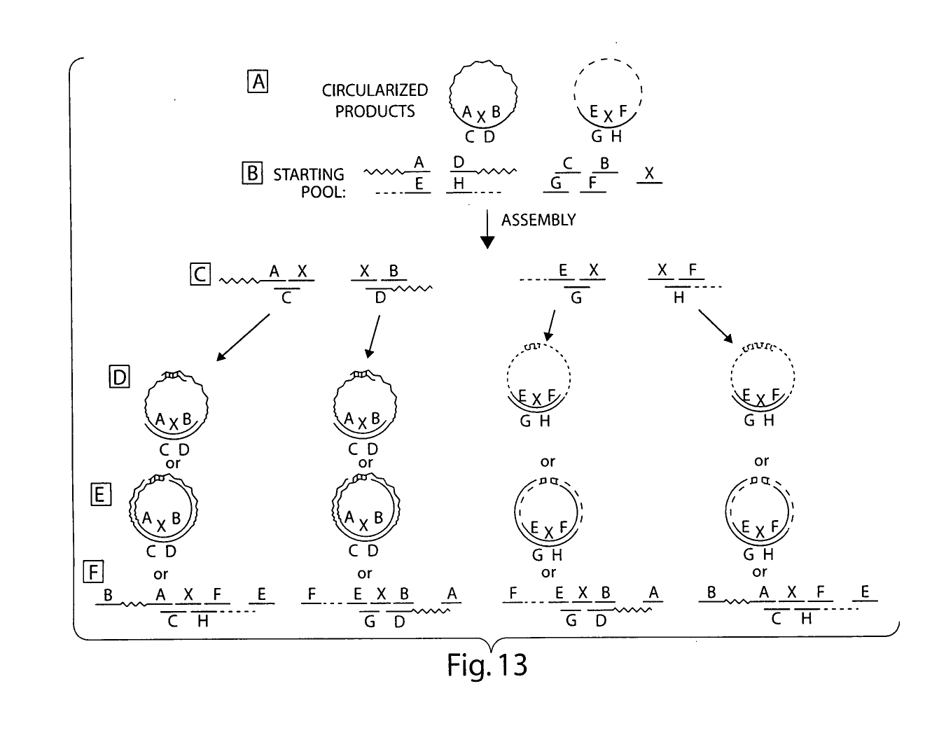

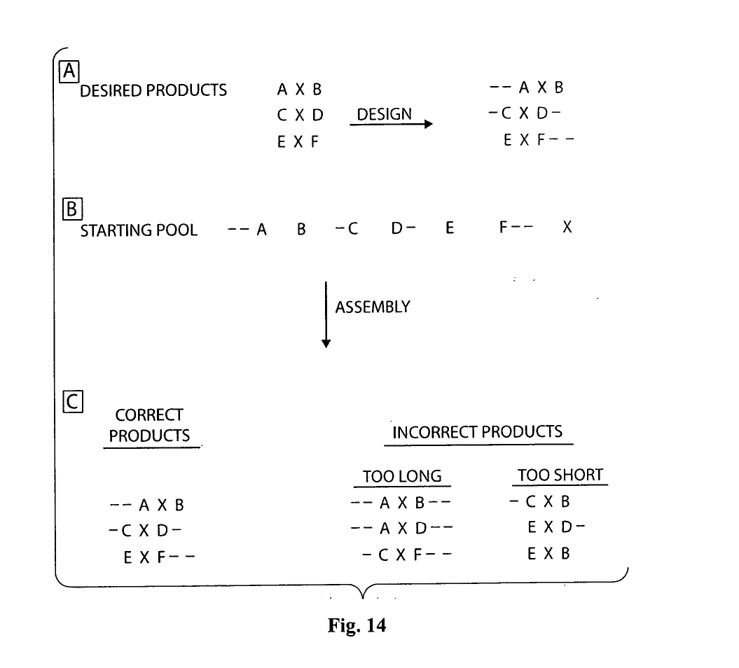

In one embodiment, undesired crossover products may be removed from a mixture of synthetic genes using the circle selection method which is illustrated in FIG. 13. The circle selection method takes advantage of the fact that circular single stranded or double stranded DNA is exonuclease resistant. FIG. 13A illustrates two polynucleotide constructs that are desired to be constructed in a single pool (represented as a single strand for purposes of illustration). As shown in FIG. 13B, the terminal construction oligonucleotides are designed to form single stranded overhangs (which may optionally be formed by designing the construction oligonucleotides to contain an appropriate linker sequence) that allow the correct polynucleotide constructs to circularize, e.g., the complementary A/C oligonucleotides form a single stranded overhang that is complementary to a single stranded overhang formed by the complementary oligonucleotides B/D (represented by wavy lines) but are not complementary to a single stranded overhang formed by the F/H oligonucleotide pair (represented by dotted lines), etc. Therefore, only the correct products may circularize, while the incorrect crossover products (e.g., B-AXF-E and F-EXB-A) remain linear and may be degraded by an exonuclease leaving the circles intact (FIGS. 13D-F). The flanking regions and circularizing segment are assembled, and then the homologous linker X is added to the mixture. The desired sequences then form circles (FIGS. 13D and 13E), while the crossover products form linear sequences (FIG. 13F). These crossover products can be selectively degraded using an exonuclease. Then, an appropriate enzyme (e.g., a restriction enzyme or uracil DNA glycosylase (UDG)) can be added to linearize the circles and/or remove the circularizing segment (linkers), leaving only the desired products, e.g., AXB and EXF (represented by top strand only). As shown in FIGS. 13D and 13E, the circularized products may be partially double stranded (FIG. 13D) or alternatively may be completely double stranded (FIG. 13E). It is also possible to convert partially double stranded circles to fully double stranded circles using a polymerase and dNTPs.

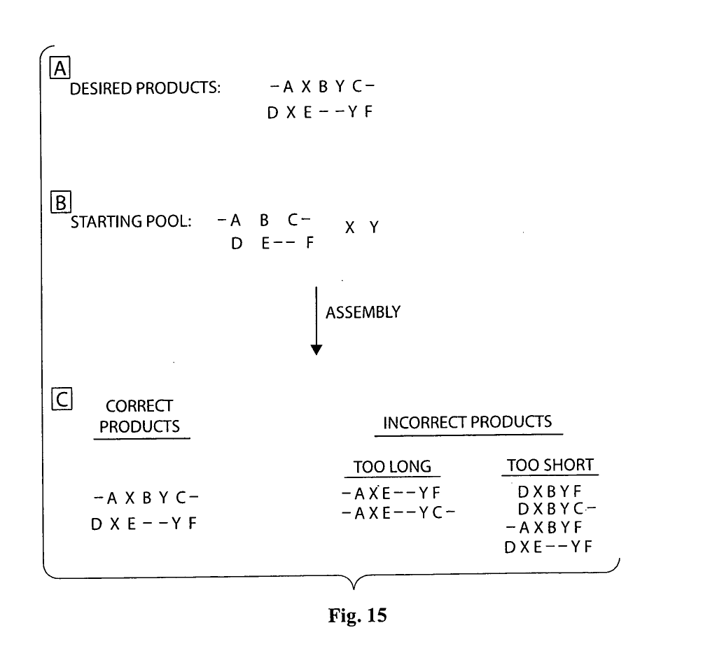

In another embodiment, undesired crossover products may be removed from a mixture of synthetic polynucleotide constructs using the size selection method which is illustrated in FIGS. 14 and 15. The size selection method takes advantage of the fact that dsDNA mobility is a function of its size, and thus DNA of different lengths can be separated, for example, via gel or column chromatography. In this embodiment, the initial genes are designed such that the desired products have different lengths than all of the crossover products (see e.g., FIGS. 14 and 15). For example, in one embodiment, the oligonucleotides are designed such that all of the desired products are about the same size, and any crossover products have significantly different sizes. This may be accomplished by designing the construction oligonucleotides such that the crossover point is in a different position in each of the target sequences. For example, as illustrated in FIG. 14, if the desired sequences are AXB, CXD, and EXF, and the A, B, C, C, E, F, and X are all approximately the same length, the sequences can be “padded” (e.g., the addition of extra bases or series of bases, represented as dashes) (FIG. 14B) to yield desired products having the same length, e.g., --AXB, -CXD-, and EXF--, and undesired crossover products having different lengths, e.g., --AXF--, --AXD-, -CXF--, -CXB, EXD-, or EXB (FIG. 14C). The polynucleotide constructs can be assembled in multiplexed format and the desired products separated from the crossover products by size selection. The padding units can then be removed using a restriction enzyme or UDG. In certain embodiments, such size selection techniques may be achieved merely through careful design of the construction oligonucleotides without the need to pad the oligonucleotides, e.g., the A, B, C, C, E, F, and X are naturally different sizes and will permit the distinction between correct vs. incorrect products.

The degree of difference in length needed to distinguish the products may be determined based on the separation method to be used. For example, if the size separation will be performed by gel electrophoresis, then a separation resolution and size differential of about +/−5-10% of the full nucleic acid sequence may be reasonable.

In another embodiment, if an internal region of DNA with known markers can be selectively excised, a single size selection could be used on sequences with more than one region of homology. This embodiment is illustrated in FIG. 15 for products AXBYC and DXEYF which may be synthesized in a single pool, for example, as -AXBYC- and DXE--YF (FIG. 15A) using the construction oligonucleotides shown in FIG. 15B. Of the eight possible products (FIG. 15C), the two desired products each contain two units of padding (“-”), while the six crossover products at X or Y contain either 0, 1, 3, or 4 units of padding (FIG. 15C). The regions of internal padding may then be excised, for example, using a restriction endonuclease (e.g., a type IIS restriction endonuclease). The fragments may then be exposed to hybridization and ligation conditions to form the correct, unpadded construct.

In another embodiment, when multiple internal homologous regions are present, separate assembly and separation steps may be performed for each homologous region. The resulting gene fragments will then be unique and can be assembled via PAM. This is a “linear” strategy which scales in complexity as the number of homologous regions. As the molecule length grows, conventional methods of error-reduction become prohibitively cumbersome and costly. Set forth below are tools for dramatically reducing errors in large-scale nucleic acid synthesis.

Biological organisms have means to detect errors in their own DNA sequences, as well as repair them. One component of this system is a mismatch binding protein which can detect short regions of DNA containing a mismatch, a region where the two DNA strands are not perfectly complementary to each other. Mismatches can be the result of a point mutation, deletion, insertion, or chemical modification. For the purpose of this invention, a mismatch includes base pairs of opposing strands with sequence A-A, C-C, T-T, G-G, A-C, A-G, T-C, T-G, or the reverse of these pairs (which are equivalent, i.e. A-G is equivalent to G-A), a deletion, insertion, or other modification to one or more of the bases. The mismatch binding proteins (MMBPs) have been used commercially for the detection of mutations and genetic differences within a population (SNP genotyping), but not for the purpose of error control in designed sequences.

In an exemplary embodiment, the biosynthetic library described herein may be constructed from oligonucleotides that have been codon remapped. The term “codon remapping” refers to modifying the codon content of a nucleic acid sequence. In many embodiments, codon remapping results in a modification of the content of the nucleic acid sequence without any modification of the sequence of the polypeptide encoded by the nucleic acid. In certain embodiments, the term is meant to encompass “codon optimization” wherein the codon content of the nucleic acid sequence is modified to enhance expression in a particular cell type. In other embodiments, the term is meant to encompass “codon normalization” wherein the codon content of two or more nucleic acid sequences are modified to minimize any possible differences in protein expression that may arise due to the differences in codon usage between the sequences. In still other embodiments, the term is meant to encompass modifying the codon content of a nucleic acid sequence as a means to control the level of expression of a protein (e.g., either increases or decrease the level of expression). Codon remapping may be achieved by replacing at least one codon in the “wild-type sequence” with a different codon encoding the same amino acid that is used at a higher or lower frequency in a given cell type. For this embodiment, “wild-type” is meant to encompass sequences that have not been codon remapped whether they are true wild-type sequences or variant sequences designed using the methods described herein. In other embodiments, the term is meant to encompass “codon reassignment” wherein a cell comprises a modified tRNA and/or tRNA synthetase so that the cell inserts an amino acid in response to a codon that is different than the amino acid inserted by a wild-type cell. Furthermore, nucleotide sequences in the cell have been correspondingly modified so that polypeptide sequences encoded by the cell comprising the modified tRNA and/or tRNA synthetase are the same as the polypeptide produced in a wild-type cell.

In an exemplary embodiment, a plurality of nucleic acid molecules in a biosynthetic library may be codon normalized and/or codon optimized. Libraries of codon normalized nucleic acids will facilitate screening and/or selection of desired protein variants by minimizing experimental differences arising from variations in the levels of polypeptide expression due to codon bias (e.g., differences in enzymatic activities, binding affinities, etc.). Libraries of codon optimized nucleic acids will facilitate screening and/or selection of desired protein variants by optimizing expression in a given host cell. In an exemplary embodiment, libraries may comprise nucleic acids that have been both codon normalized and codon optimized.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, nucleic acid sequences can be tailored for optimal expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database” available on the world wide web at kazusa.orjp/codon/, and these tables can be adapted in a number of ways. See Nakamura, Y., et al. Codon usage tabulated from the international DNA sequence databases: status for the year 2000, Nucl. Acids Res. 28:292 (2000). These tables use mRNA nomenclature, and so instead of thymine (T) which is found in DNA, the tables use uracil (U) which is found in RNA. The tables have been adapted so that frequencies are calculated for each amino acid, rather than for all 64 codons.

By utilizing these or similar tables, one of ordinary skill in the art can apply the frequencies to any given polypeptide sequence, and produce a nucleic acid fragment of a codon remapped coding region which encodes the same polypeptide, but which uses codons more or less optimal for a given species.

Codon remapped coding regions can be designed by various methods. For example, codon optimization may be carried out using a method termed “uniform optimization” wherein a codon usage table is used to find the single most frequent codon used for any given amino acid, and that codon is used each time that particular amino acid appears in the polypeptide sequence. For example, in humans the most frequent leucine codon is CUG, which is used 41% of the time. Therefore, codon optimization may be carried out by assigning the codon CUG for all leucine residues in a given amino acid.

In another method, termed “full-optimization,” the actual frequencies of the codons are distributed randomly throughout the coding region. Thus, using this method for optimization, if a hypothetical polypeptide sequence had 100 leucine residues and was to be optimized for expression in human cells, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence can vary significantly using this method, however, the sequence always encodes the same polypeptide. Such methods may be adapted similarly adapted for other codon remapping techniques, including codon normalization.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG—Wisconsin Package, available from Accelrys, Inc., San Diego, Calif. In addition, various resources are publicly available to codon-optimize coding region sequences. For example, the “backtranslation” function on the world wide web at entelechon.com/eng/backtranslation.html, the “backtranseq” function available on the world wide web at bioinfo.pbi.nrc.ca:-8090/EMBOSS/index.html. Constructing a rudimentary algorithm to assign codons based on a given frequency can also easily be accomplished with basic mathematical functions by one of ordinary skill in the art.

In various embodiments, mismatch binding proteins can be used to control the errors generated during oligonucleotide synthesis, polynucleotide assembly, and the construction of nucleic acids of different sizes. Though biological systems use this function when synthesizing DNA, it requires the presence of a template strand. For de novo synthesis, as employed by this technique, one is starting by definition without a template.

When attempting to produce a desired DNA molecule, a mixture typically results containing some correct copies of the sequence, and some containing one or more errors. But if the synthetic oligonucleotides are annealed to their complementary strands of DNA (also synthesized), then a single error at that sequence position on one strand will give rise to a base mismatch, causing a distortion in the DNA duplex. These distortions can be recognized by a mismatch binding protein. One example of such a protein is mutS from the bacterium Escherichia coli. Once an error is recognized, a variety of possibilities exist for how to prevent the presence of that error in the final desired DNA sequence.

When using pairs of complementary DNA strands for error recognition, each strand in the pair may contain errors at some frequency, but when the strands are annealed together, the chance of errors occurring at a correlated location on both strands is very small, with an even smaller chance that such a correlation will produce a correctly matched Watson-Crick base pair (e.g. A-T, G-C). For example, in a pool of 50-mer oligonucleotides, with a per-base error rate of 1%, roughly 60% of the pool (0.9950) will have the correct sequence, and the remaining forty percent will have one or more errors (primarily one error per oligonucleotide) in random positions. The same would be true for a pool composed of the complementary 50-mer. After annealing the two pools, approximately 36% (0.62) of the DNA duplexes will have correct sequence on both strands, 48% (2×0.4×0.6) will have an error on one strand, and 16% (0.42) will have errors in both strands. Of this latter category, the chance of the errors being in the same location is only 2% ( 1/50) and the chance of these errors forming a Watson-Crick base pair is even less (⅓× 1/50). These correlated mismatches, which would go undetected, then comprise 0.11% of the total pool of DNA duplexes (16×⅓× 1/50). Removal of all detectable mismatch-containing sequences would thus enrich the pool for error-free sequences (i.e. reduce the proportion of error-containing sequences) by a factor of roughly 200 (0.6/0.4 originally for the single strands vs. 0.36/0.0011 after mismatch detection and removal). Furthermore, the remaining oligonucleotides can then be dissociated and re-annealed, allowing the error-containing strands to partner with different complementary strands in the pool, producing different mismatch duplexes. These can also be detected and removed as above, allowing for further enrichment for the error-free duplexes. Multiple cycles of this process can in principle reduce errors to undetectable levels. Since each cycle of error control may also remove some of the error-free sequences (while still proportionately enriching the pool for error-free sequences), alternating cycles of error control and DNA amplification can be employed to maintain a large pool of molecules.

In one embodiment, the number of errors detected and corrected may be increased by melting and reannealing a pool of DNA duplexes prior to error reduction. For example, if the DNA duplexes in question have been amplified by a technique such as the polymerase chain reaction (PCR) the synthesis of new (perfectly) complementary strands would mean that these errors are not immediately detectable as DNA mismatches. However, melting these duplexes and allowing the strands to re-associate with new (and random) complementary partners would generate duplexes in which most errors would be apparent as mismatches, as described above.

Many of the methods described below can be used together, applying error-reducing steps at multiple points along the way to produce a long nucleic acid molecule. Error reduction can be applied to the first oligonucleotide duplexes generated, then for example to intermediate 500-mers or 1000-mers, and then even to larger full length nucleic acid sequences of 10,000-mers or more. In an exemplary embodiment, the methods described herein may be used to produce the entire genome of an organism optionally incorporating specific modifications into the sequence at one or more desired locations.

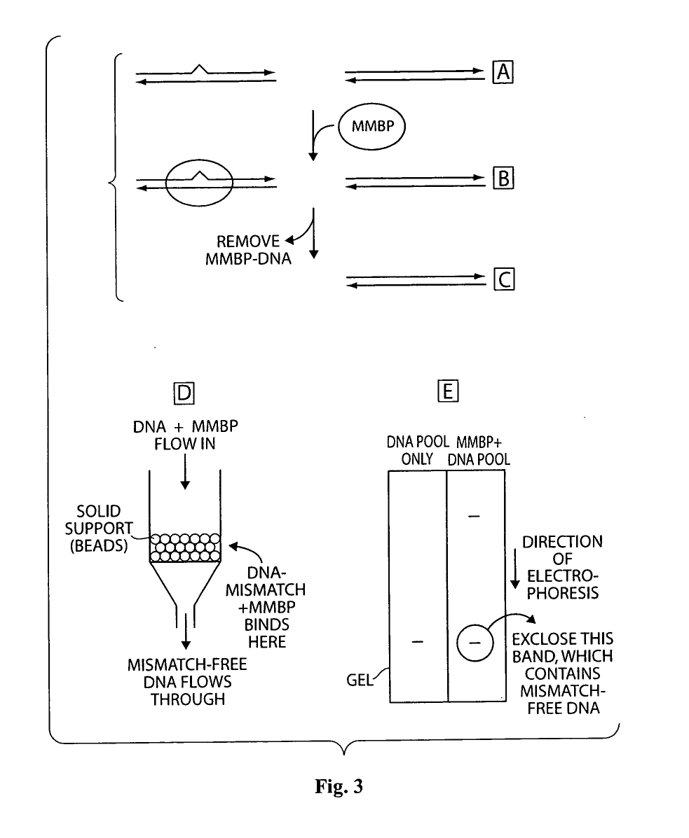

FIG. 3 illustrates an exemplary method for removing sequence errors using mismatch binding proteins. An error in a single strand of DNA causes a mismatch in a DNA duplex. A mismatch binding protein (MMBP), such as a dimer of mutS, binds to this site on the DNA. As shown in FIG. 3A, a pool of DNA duplexes contains some duplexes with mismatches (left) and some which are error-free (right). The 3′-terminus of each DNA strand is indicated by an arrowhead. An error giving rise to a mismatch is shown as a raised triangular bump on the top left strand. As shown in FIG. 3B, a MMBP may be added which binds selectively to the site of the mismatch. The MMBP-bound DNA duplex may then be removed, leaving behind a pool which is dramatically enriched for error-free duplexes (FIG. 3C). In one embodiment, the DNA-bound protein provides a means to separate the error-containing DNA from the error-free copies (FIG. 3D). The protein-DNA complexes can be captured by affinity of the protein for a solid support functionalized, for example, with a specific antibody, immobilized nickel ions (protein is produced as a his-tag fusion), streptavidin (protein has been modified by the covalent addition of biotin) or other such mechanisms as are common to the art of protein purification. Alternatively, the protein-DNA complex is separated from the pool of error-free DNA sequences by a difference in mobility, for example, using a size-exclusion column chromatography or by electrophoresis (FIG. 3E). In this example, the electrophoretic mobility in a gel is altered upon MMBP binding: in the absence of MMBP all duplexes migrate together, but in the presence of MMBP, mismatch duplexes are retarded (upper band). The mismatch-free band (lower) is then excised and extracted.

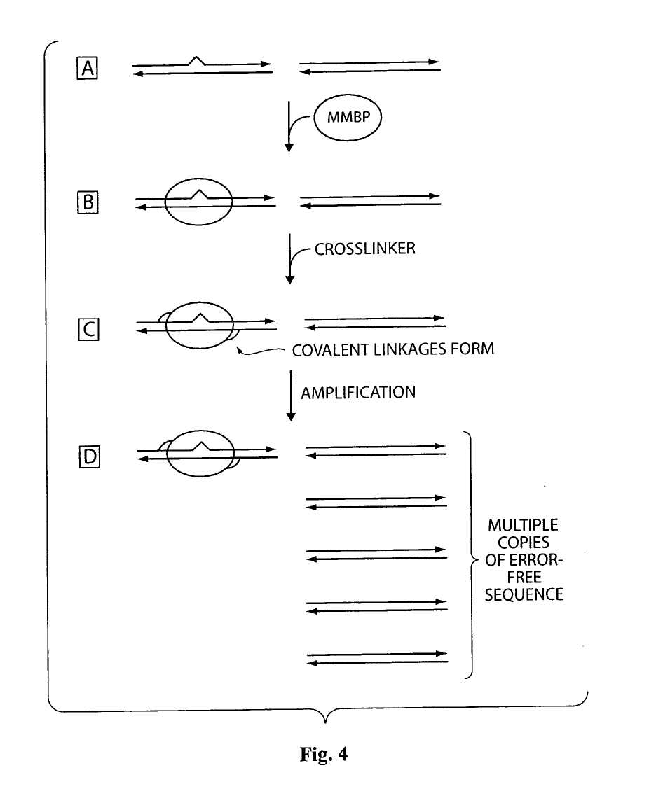

FIG. 4 illustrates an exemplary method for neutralizing sequence errors using a mismatch binding protein. In this embodiment, the error-containing DNA sequence is not removed from the pool of DNA products. Rather, it becomes irreversibly complexed with a mismatch recognition protein by the action of a chemical crosslinking agent (for example, dimethyl suberimidate, DMS), or of another protein (such as mutL). The pool of DNA sequences is then amplified (such as by the polymerase chain reaction, PCR), but those containing errors are blocked from amplification, and quickly become outnumbered by the increasing error-free sequences. FIG. 4A illustrates an exemplary pool of DNA duplexes containing some duplexes with mismatches (left) and some which are error-free (right). A MMBP may be used to bind selectively to the DNA duplexes containing mismatches (FIG. 4B). The MMBP may be irreversibly attached at the site of the mismatch upon application of a crosslinking agent (FIG. 4C). In the presence of the covalently linked MMBP, amplification of the pool of DNA duplexes produces more copies of the error-free duplexes (FIG. 4D). The MMBP-mismatch DNA complex is unable to participate in amplification because the bound protein prevents the two strands of the duplex from dissociating. For long DNA duplexes, the regions outside the MMBP-bound site may be able to partially dissociate and participate in partial amplification of those (error-free) regions.

As increasingly longer sequences of DNA are generated, the fraction of sequences which are completely error-free diminishes. At some length, it becomes likely that there will be no molecule in the entire pool which contains a completely correct sequence. Thus, for the generation of extremely long segments of DNA, it can be useful to produce smaller units first which can be subjected to the above error control approaches. Then these segments can be combined to yield the larger full length product. However, if errors in these extremely long sequences can be corrected locally, without removing or neutralizing the entire long DNA duplex, then the more complex stepwise assembly process can be avoided.

Many biological DNA repair mechanisms rely on recognizing the site of a mutation (error) and then using a template strand (most likely error-free) to replace the incorrect sequence. In the de novo production of DNA sequences, this process is complicated by the difficulty of determining which strand contains the error and which should be used as the template. One solution to this problem relies on using the pool of other sequences in the mixture to provide the template for correction. These methods can be very robust: even if every strand of DNA contains one or more errors, as long as the majority of strands have the correct sequence at each position (expected because the positions of errors are generally not correlated between strands), there is a high likelihood that a given error will be replaced with the correct sequence. FIGS. 5-10 present exemplary procedures for performing this sort of local error correction.

FIG. 5 illustrates an exemplary method for carrying out strand-specific error correction. In replicating organisms, enzyme-mediated DNA methylation is often used to identify the template (parent) DNA strand. The newly synthesized (daughter) strand is at first unmethylated. When a mismatch is detected, the hemimethylated state of the duplex DNA is used to direct the mismatch repair system to make a correction to the daughter strand only. However, in the de novo synthesis of a pair of complementary DNA strands, both strands are unmethylated, and the repair system has no intrinsic basis for choosing which strand to correct. Methylation and site-specific demethylation are employed to produce DNA strands that are selectively hemi-methylated. A methylase, such as the Dam methylase of E. coli, is used to uniformly methylate all potential target sites on each strand. The DNA strands are then dissociated, and allowed to re-anneal with new partner strands. A new protein is applied, a fusion of a mismatch binding protein (MMBP) with a demethylase. This fusion protein binds only to the mismatch, and the proximity of the demethylase removes methyl groups from either strand, but only near the site of the mismatch. A subsequent cycle of dissociation and annealing allows the (demethylated) error-containing strand to associate with a (methylated) strand which is error-free in this region of its sequence. (This should be true for the majority of the strands, since the locations of errors on complementary strands are not correlated.) The hemi-methylated DNA duplex now contains all the information needed to direct the repair of the error, employing the components of a DNA mismatch repair system, such as that of E. coli, which employs mutS, mutL, mutH, and DNA polymerase proteins for this purpose. The process can be repeated multiple times to ensure all errors are corrected.

FIG. 5A shows two DNA duplexes that are identical except for a single base error in the top left strand, giving rise to a mismatch. The strands of the right hand duplex are shown with thicker lines. Methylase (M) may then be used to uniformly methylate all possible sites on each DNA strand (FIG. 5B). The methylase is then removed, and a protein fusion is applied, containing both a mismatch binding protein (MMBP) and a demethylase (D) (FIG. 5C). The MMBP portion of the fusion protein binds to the site of the mismatch thus localizing the fusion protein to the site of the mismatch. The demethylase portion of the fusion protein may then act to specifically remove methyl groups from both strands in the vicinity of the mismatch (FIG. 5D). The MMBP-D protein fusion may then be removed, and the DNA duplexes may be allowed to dissociated and re-associate with new partner strands (FIG. 5E). The error-containing strand will most likely re-associate with a complementary strand which a) does not contain a complementary error at that site; and b) is methylated near the site of the mismatch. This new duplex now mimics the natural substrate for DNA mismatch repair systems. The components of a mismatch repair system (such as E. coli mutS, mutL, mutH, and DNA polymerase) may then be used to remove bases in the error-containing strand (including the error), and uses the opposing (error-free) strand as a template for synthesizing the replacement, leaving a corrected strand (FIG. 5F).

FIG. 6 illustrates an exemplary method for local removal of DNA on both strands at the site of a mismatch. Various proteins can be used to create a break in both DNA strands near an error. For example, an MMBP fusion to a non-specific nuclease (such as DNAseI) can direct the action of the nuclease (N) to the mismatch site, cleaving both strands. Once the break is generated, homologous recombination can be employed to use other strands (most of which will be error-free at this site) as template to replace the excised DNA. For example, the RecA protein can be used to facilitate single strand invasion, and early step in homologous recombination. Alternatively, a polymerase can be employed to allow broken strands to reassociate with new full-length partner strands, synthesizing new DNA to replace the error. For example, FIG. 6A shows two DNA duplexes that identical except that one contains a single base error as in FIG. 5A. In one embodiment, a protein, such as a fusion of a MMBP with a nuclease (N), may be added and will bind at the site of the mismatch (FIG. 6B). Alternatively, a nuclease with specificity for single-stranded DNA can be employed, using elevated temperatures to favor local melting of the DNA duplex at the site of the mismatch. (In the absence of a mismatch, a perfect DNA duplex will be less likely to melt.) An endonuclease, such as that of the MMBP-N fusion, may be used to make double-stranded breaks near the site of the mismatch (FIG. 6C). The MMBP-N complex is then removed, along with the bound short region of DNA duplex around the mismatch (FIG. 6D). Melting and re-annealing of partner strands produces some duplexes with single-stranded gaps. A DNA polymerase may then be used to fill in the gaps, producing DNA duplexes without the original error (FIG. 6E).

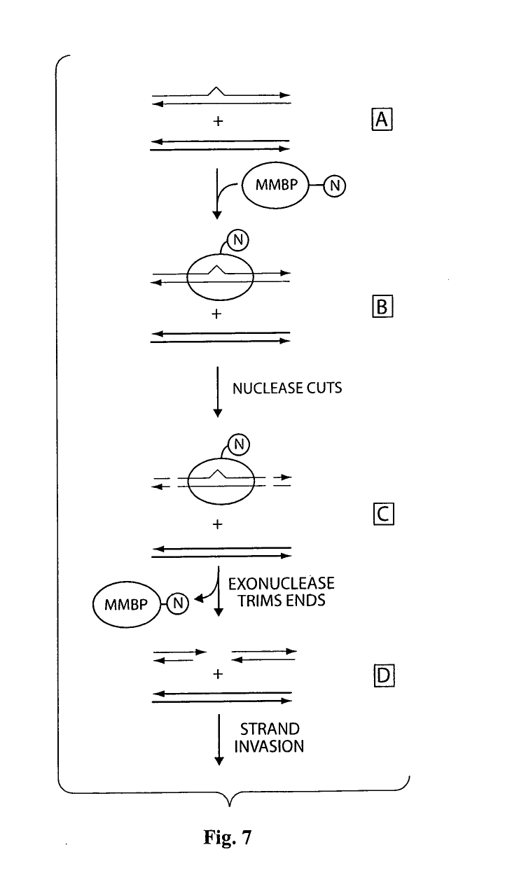

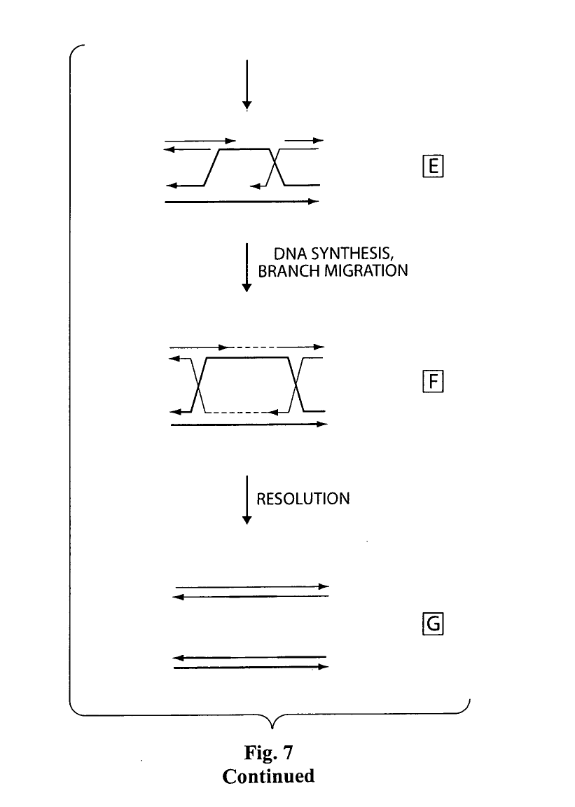

FIG. 7 illustrates a process similar to that of FIG. 6, however, in this embodiment, double-stranded gaps in DNA duplexes are repaired using the protein components of a recombination repair pathway. (Note that in this case no global melting and re-annealing of DNA strands is required, which can be preferable when dealing with especially large DNA molecules, such as genomic DNA.) For example, FIG. 7A shows two DNA duplexes (as in FIG. 6A), identical except that one contains a single base mismatch. As in FIG. 6B, a protein, such as a fusion of a MMBP with a nuclease (N), is added to bind at the site of the mismatch (FIG. 7B). As in FIG. 6C, an endonuclease, such as that of the MMBP-N fusion, may be used to make double-stranded breaks around the site of the mismatch (FIG. 7C). Protein components of a DNA repair pathway, such as the RecBCD complex, may then be employed to further digest the exposed ends of the double-stranded break, leaving 3′ overlaps (FIG. 7D). Subsequently, protein components of a DNA repair pathway, such as the RecA protein, are employed to facilitate single strand invasion of the intact DNA duplex, forming a Holliday junction (FIG. 7E). A DNA polymerase may then be used to synthesize new DNA, filling in the single-stranded gaps (FIG. 7F). Finally, protein components of a DNA repair pathway may be employed, such as the RuvC protein, to resolve the Holliday junction (FIG. 7G). The two resulting DNA duplexes do not contain the original error. Note that there can be more than one way to resolve such junctions, depending on migration of the branch points.

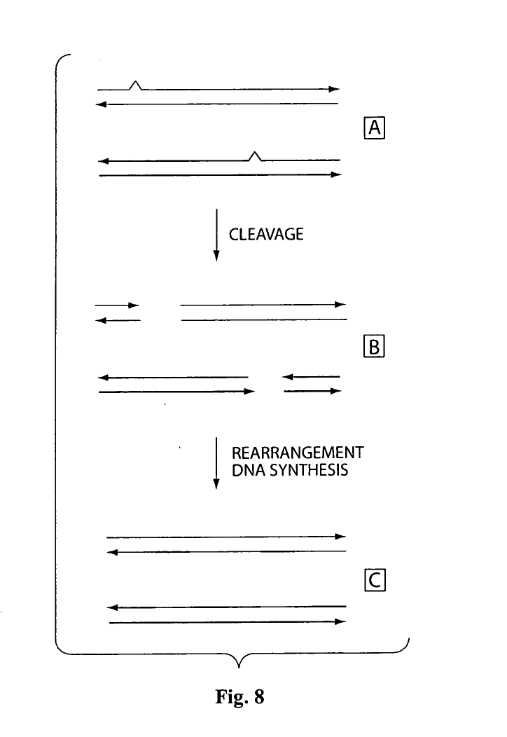

It is important to make clear that the methods described herein are capable of generating large error-free DNA sequences, even if none of the initial DNA products are error-free. FIG. 8 summarizes the effects of the methods of FIG. 6 (or equivalently, FIG. 7) applied to two DNA duplexes, each containing a single base (mismatch) error. For example, FIG. 8A illustrates two DNA duplexes, identical except for a single base mismatch in each, at different locations in the DNA sequence. Mismatch binding and localized nuclease activity are then used to generate double-stranded breaks which excise the errors (FIG. 8B). Recombination repair (as in FIG. 7) or melting and reassembly (as in FIG. 6) are employed to generate DNA duplexes where each excised error sequence has been replaced with newly synthesized sequence, each using the other DNA duplex as template (and unlikely to have an error in that same location) (FIG. 8C). Note that complete dissociation and re-annealing of the DNA duplexes is not necessary to generate the error-free products (if the methods shown in FIG. 7 are employed).

A simple way to reduce errors in long DNA molecules is to cleave both strands of the DNA backbone at multiple sites, such as with a site-specific endonuclease which generates short single stranded overhangs at the cleavage site. Of the resulting segments, some are expected to contain mismatches. These can be removed by the action and subsequent removal of a mismatch binding protein, as described in FIG. 3. The remaining pool of segments can be re-ligated into full length sequences. As with the approach of FIG. 7, this approach includes several advantages such as: 1) removal of an entire full length DNA duplex is not required to remove an error; 2) global dissociation and re-annealing of DNA duplexes is not necessary; 3) error-free DNA molecules can be constructed from a starting pool in which no one member is an error-free DNA molecule.

If the most common type of restriction endonuclease were employed for this approach, all DNA cleavage sites would result in identical overhangs. Thus the segments would associate and ligate in random order. However, use of a site-specific “outside cutter” endonuclease (such as HgaI, FokI, or BspMI) produces cleavage sites adjacent to (non-overlapping) the DNA recognition site. Thus each overhang would have sequence specific to that part of the DNA, distinct from that of the other sites. The re-association of these specifically complementary cohesive ends will then cause the segments to come together in the proper order. The cohesive ends generated can be up to five bases in length, allowing for up to 45=1024 different combinations. Conceivably this many distinct restriction sites could be employed, though the need to avoid near matches between cohesive ends could lower this number.

The necessary restriction sites can be specifically included in the design of the sequence, or the random distribution of restriction sites within a desired sequence can be utilized (the recognition sequence of each endonuclease allows prediction of the typical distribution of fragments produced). Also, the target sequence can be analyzed for which choice of endonuclease produces the most ideal set of fragments.

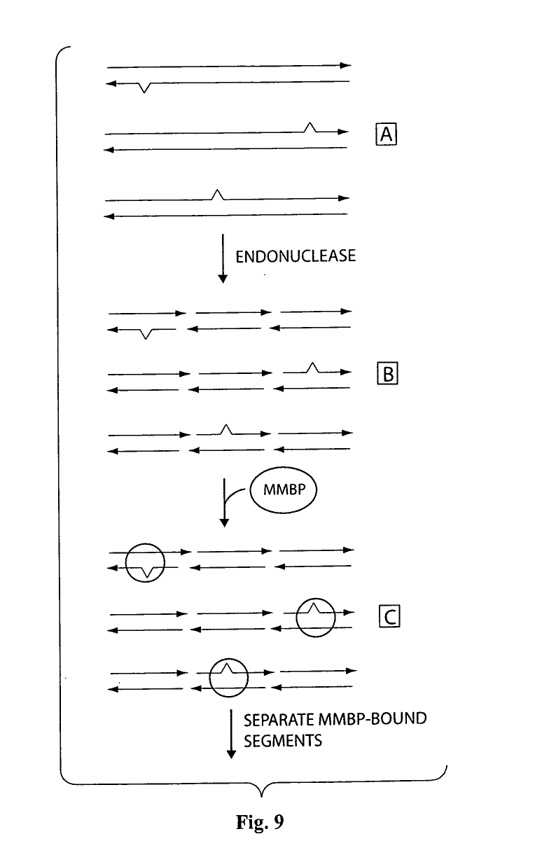

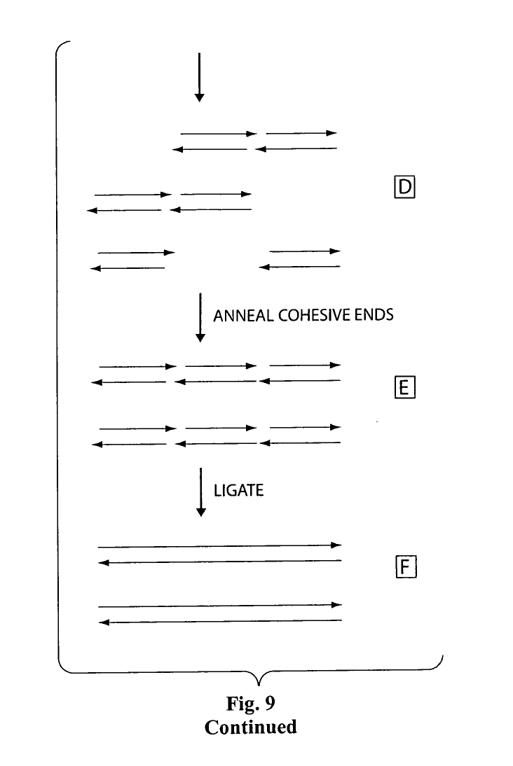

FIG. 9 shows an example of semi-selective removal of mismatch-containing segments. For example, FIG. 9A illustrates three DNA duplexes, each containing one error leading to a mismatch. The DNA is cut with a site-specific endonuclease, leaving double-stranded fragments with cohesive ends complementary to the adjacent segment (FIG. 9B). A MMBP is then applied, which binds to each fragment containing a mismatch (FIG. 9C). Fragments bound to MMBP are removed from the pool, as described in FIG. 3 (FIG. 9D). The cohesive ends of each fragment allow each DNA duplex to associate with the correct sequence-specific neighbor fragment (FIG. 9E). A ligase (such T4 DNA ligase) is employed to join the cohesive ends, producing full length DNA sequences (FIG. 9F). These DNA sequences can be error-free in spite of the fact that none of the original DNA duplexes was error-free. Incomplete ligation may leave some sequences which are less than full-length, which can be purified away on the basis of size.

The above approaches provide a major advantage over one of the conventional methods of removing errors, which employs sequencing first to find an error, and then relies on choosing specific error-free subsequences to “cut and paste” with endonuclease and ligase. In this embodiment, no sequencing or user choice is required in order to remove errors.

When complementary DNA strands are synthesized and allowed to anneal, both strands may contain errors, but the chance of errors occurring at the same base position in both sequences is extremely small, as discussed above. The above methods are useful for eliminating the majority of cases of uncorrelated errors which can be detected as DNA mismatches. In the rare case of complementary errors at identical positions on both strands (undetectable by the mismatch binding proteins), a subsequent cycle of duplex dissociation and random re-annealing with a different complementary strand (with a different distribution of error positions) remedies the problem. But in some applications it is desirable to not melt and re-anneal the DNA duplexes, such as in the case of genomic-length DNA strands. In such an embodiment, correlated errors may be removed using a different method. For example, though the initial population of correlated errors is expected to be low, amplification or other replication of the DNA sequences in a pool will ensure that each error is copied to produce a perfectly complementary strand which contains the complementary error. This approach does not require global dissociation and re-annealing of the DNA strands. Essentially, various forms of DNA damage and recombination are employed to allow single-stranded portions of the long DNA duplex to re-assort into different duplexes.

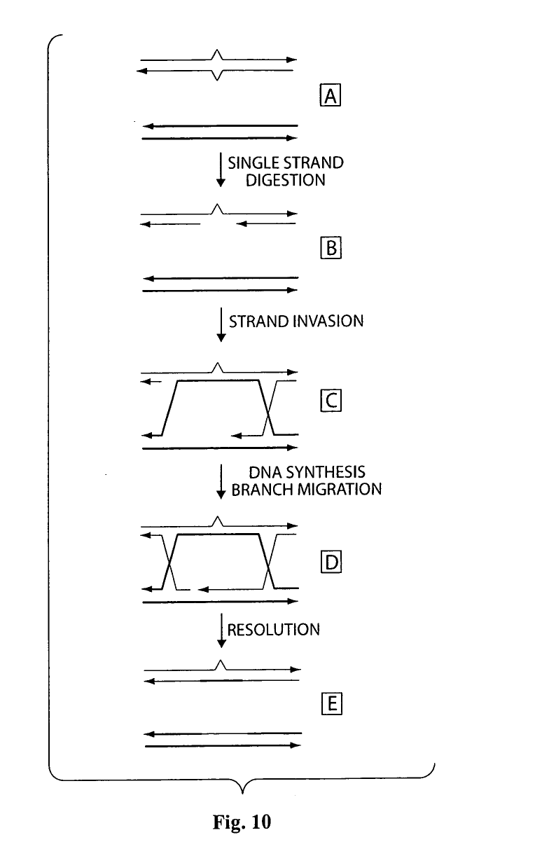

FIG. 10 shows a procedure for reducing correlated errors in synthesized DNA. FIG. 10A shows two DNA duplexes identical except for a single error in one strand. Non-specific nucleases may be used to generate short single-stranded gaps in random locations in the DNA duplexes in the pool (FIG. 10B). Shown here is the result of one of these gaps generated at the site of one of the correlated locations. Recombination-specific proteins such as RecA and RuvB are employed to mediate the formation of a four-stranded Holliday junction (FIG. 10C). DNA polymerase is employed to fill in the gap shown in the lower portion of the complex (FIG. 10D). Action of other recombination and/or repair proteins such as RuvC is employed to cleave the Holliday junction, resulting in two new DNA duplexes, containing some sequences which are hybrids of their progenitors (FIG. 10E). In the example shown, one of the error-containing regions has been eliminated. However, since the cutting, rearrangement, and replacement of strands employed in this method is intended to be random, it is expected that the total number of errors in the sequence will actually not change, simply that errors will be reasserted to different strands. Thus, pairs of errors correlated in one duplex will be reshuffled into separate duplexes, each with a single error. This random reassortment of strands will yield new duplexes containing mismatches which can be repaired using the mismatch repair proteins detailed above. Unique to this embodiment is the use of recombination to separate the correlated errors into different DNA duplexes.

This process makes possible the direct fabrication of DNA of any desired sequence. No longer do expression vectors have to be constructed from component parts by techniques of in vitro recombinant DNA. Instead, any desired DNA construct can be directly synthesized in total by direct synthesis in segments followed by spontaneous assembly into the completed molecule. The constructed DNA molecule does not have to be one that previously existed, it can be a totally novel construct to suit a particular purpose. It now becomes possible for one of skill in the art to design a desired DNA sequence or vector entirely in the computer, and then to directly synthesize the DNA vector artificially in a single operation.

It is envisioned that the process of direct DNA synthesis envisioned here will begin with a desired target DNA sequence, in the form of a computer file representing the target sequence that the user wants to build. A computer software program is used to determine the optimal way to subdivide the desired DNA construct into smaller DNA that can be used to build the larger target sequence. The software would be optimized for this purpose. For example, the target DNA construct should be subdivided into segments in such a manner so that the hybridizing half of each segment will hybridize well to a corresponding half segment, and not to any other half segment. If needed, changes to the sequence not affecting the ultimate functionality of the DNA may be required in some instances to ensure unique segments. This sort of optimization is preferable done by computer systems designed for this purpose.

After the DNA segments are constructed on the substrate of the microarray, the DNA segments must be separated from the microarray substrate. This can be done by any of a number of techniques, depending on the technique used to attach the DNA segments to the substrate in the first place. Described below is one technique based on base labile chemistry, adapted from techniques used to fabricate oligonucleotides on glass particles, but this is only one example among several possibilities. In essence, all that is required is that the attachment of the DNA segments to the substrate be cleaved by a technique that does not destroy the DNA molecules themselves.

This process may or may not make enough directly synthesized DNA as needed for a particular application. It is envisioned that more copies of the synthesized DNA can be made by any of the several ways in which other DNA constructs are cloned or replicated in quantity. An origin of replication can be built into circular DNA which would permit the rapid amplification of copies of the constructed DNA in a bacterial host. Linear DNA can be constructed with defined DNA primers at each end which can then be used to amplify many copies of the DNA construct by the PCR process.

4. Selection of Novel Proteins with Desired Characteristics

In another aspect, the invention provides methods for producing a protein having a desired characteristic or property comprising: generating sequence data for a plurality of possible proteins; in parallel, assembling a plurality of library polynucleotides from the above described library to produce polynucleotide constructs that encode at least 10 of the proteins; expressing the polynucleotide constructs to produce the proteins; and selecting or screening the proteins to identify those species having one or more desired characteristics using a high throughput assay. In an exemplary embodiment, the method involves assembling the polynucleotide constructs using hybridization of complementary construction oligonucleotides followed by ligase and/or polymerase treatment, and producing at least 20, 50, 100, 10³, 10⁴, 10⁵, or 10⁶ of the proteins. Alternatively, polynucleotides encoding the peptides of a peptide sequence library and appropriate junction nucleic acids could be produced to permit PCR based assembly of a combinatorial library of polynucleotide constructs encoding a plurality of novel proteins. The polynucleotide constructs may then be expressed to produce a library of proteins. Proteins produced by such methods are then assayed for one or more desired function or property, using assays known for such function or property. Alternatively, the methods may involve construction of large nucleic molecules with high fidelity using stepwise assembly of complementary, overlapping, construction oligonucleotides. In exemplary embodiments, at least 10, 100, 1,000, 10,000, 100,000 or more designed proteins are experimentally tested. Once a protein having a desired characteristics is identified, it may be produced in useful quantities by any method known in the art. In a preferred embodiment, the production process does not introduce post-translational modifications that could stimulate an immune response in humans. Examples of post-translation modifications include, glycosylation, acylation, phosphorylation, methylation, sulfation and prenylation.

In some embodiments, an initial screening step may be conducted in silico, wherein the predicted structures of proteins assembled from the peptides of the peptide sequence library are compared with a naturally occurring protein possessing a desired characteristic. Novel proteins that share structural elements correlating with the desired characteristic are selected as candidate proteins. These candidate proteins are then expressed from synthetic polynucleotides and tested for the desired characteristic. The proteins exhibiting the desired characteristic will then be selected and produced as described herein.

In exemplary embodiments, a variety of novel proteins selected from a protein library may be expressed and further screened to identify proteins that exhibit one or more desired characteristics. Selection protocols are preferred over screening protocols because of their much more efficient throughput rate, but both techniques can be used in an appropriate situation. Screening involves the assessment of a given construct for one or more properties of interest; selection involves retrieving or isolating species from a multispecies library that have a particular property, e.g., panning, as is used in phage or ribosomal display. In one embodiment, the novel proteins may be expressed using an in vitro transcription and/or translation system. In another embodiment, nucleic acids encoding the novel proteins may be inserted into an expression vector and introduced into a cell for protein expression and screening or selection. Suitable methods for screening and selection for a biochemical characteristic of a novel protein include, for example, in vitro or in vivo assays for enzymatic activity or binding interactions (including protein/protein, protein/small molecule, etc.).

Before expressing the novel library proteins, certain modifications can be made. For example, the library protein may be made as a fusion protein, perhaps to produce a dual-activity or multi-activity protein by fusing the library protein to another protein. Alternatively, the fusion protein may be created to increase expression, or for other reasons. For example, a nucleic acid encoding a library protein may be linked to other nucleic acid sequences for expression purposes. Similarly, other fusion partners may be used, such as targeting sequences which allow the localization of the library members into a subcellular or extracellular compartment of the cell, rescue sequences or purification tags which allow purification or isolation of either the library protein or the nucleic acids encoding them; stability sequences, which confer stability or protection from degradation to the library protein or the nucleic acid encoding it, for example resistance to proteolytic degradation, or combinations of these, as well as linker sequences as needed.

Examples of suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred is localization to either subcellular locations or to the outside of the cell via secretion.

In a preferred embodiment, the library member comprises a rescue sequence. A rescue sequence is a sequence which may be used to purify or isolate either the library protein or the nucleic acid encoding it. Exemplary peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST. Alternatively, the rescue sequence may be a unique nucleic acid sequence which serves as a probe target site to allow quick and easy isolation of the nucleic acid construct, via PCR, related techniques, or hybridization.

In a preferred embodiment, the fusion partner is a stability sequence to confer stability to the library protein or the nucleic acid encoding it. Thus, for example, polypeptides may be stabilized by the incorporation of glycines after the initiation methionine (MG or MGG), for protection of the polypeptide from ubiquitination as per Varshavsky's N-End Rule, thus conferring longer half-life in the cytoplasm. Similarly, two prolines at the C-terminus produce polypeptides that are largely resistant to carboxypeptidase action. The presence of two glycines prior to the prolines impart both flexibility and prevent structure initiating events in the di-proline from being propagated into the candidate protein structure. Thus, preferred stability sequences are as follows: MG(X)ₙGGPP, where X is any amino acid and n is an integer of at least four.

These fusion proteins may be cleaved at the site of fusion to restore the unmodified library protein from the fusion protein after expression. For example, the rescue sequence can be cleaved after purification is accomplished.

In one embodiment, the library nucleic acids and proteins of the invention are labeled. For example, nucleic acids and proteins may be modified with a detectable label, such as, for example, an element, isotope or chemical compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

Expression Vectors

In one embodiment, the nucleic acids of the present invention may be incorporated into an expression vector. The expression vectors may be either self-replicating extrachromosomal vectors or vectors which integrate into a host genome. Generally, these expression vectors include transcriptional and translational regulatory nucleic acid sequences operably linked to the nucleic acid encoding the library protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid sequences will generally be appropriate to the host cell used to express the library protein, as will be appreciated by those in the art. Merely for purposes of illustration, transcriptional and translational regulatory nucleic acid sequences, for example, from Bacillus are preferably used to express the library protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Promoter sequences include constitutive and inducible promoter sequences. The promoters may be either naturally occurring promoters, hybrid or synthetic promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

In addition, in a preferred embodiment, the expression vector contains a selection gene to permit the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a product that confers resistance to a selection agent. Suitable selection agents include, but are not limited to, neomycin (or its analog G418), blasticidin S, histinidol D, bleomycin, puromycin, hygromycin B, and other drugs.

In a preferred embodiment, the expression vector contains an RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988. A preferred expression vector system is a retroviral vector system such as is generally described in Mann et al., Cell, 33:153 (1993); Pear et al., Proc. Natl. Acad. Sci. USA, 90:8392 (1993); Kitamura et al., Proc. Natl. Acad. Sci. US., 92:9146 (1995); Kinsella et al., Human Gene Therapy, 7:1405 (1996); Hofmann et al., Proc. Natl. Acad. Sci. USA, 93:5185 (1996); Choate et al., Human Gene Therapy, 7:2247 (1996); PCT Publication Nos. WO 97/27212 and WO 97/27213, and references cited therein, all of which are hereby expressly incorporated by reference.

Cellular Expression Systems

The library proteins of the present invention may be produced by culturing a host cell comprising a nucleic acid (such as an expression vector) encoding a library protein under the appropriate conditions to induce or cause expression of the library protein. The conditions appropriate for library protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest may be important. For example, the baculoviral systems used in insect cell expression are lytic viruses, and thus harvest time selection can be crucial for product yield.

Transforming cells with a library of novel protein sequences will yield a plurality of cells carrying the library of novel proteins, which may be considered a cellular library. Thus, in one embodiment, the methods of the present invention comprise introducing a nucleic acid library into a plurality of cells to create a cellular library.

As will be appreciated by those in the art, the type of cells used in the present invention can vary widely. Basically, a wide variety of appropriate host cells can be used, including animal cells, in particular mammalian cells, insect cells, yeast, bacteria, archaebacteria, and fungi, which are further described below. Of particular interest are human cells, including primary cultures of isolated cells from all tissue and organ sources and also immortalized and/or transformed cells.

Mammalian systems. In a preferred embodiment, the library proteins are expressed in mammalian cells. Any mammalian cells may be used, with mouse, rat, primate and human cells being particularly preferred. Cells of human origin are the most preferred, although as will be appreciated by those in the art, modifications of the system by pseudotyping allows all eukaryotic cells to be used, preferably higher eukaryotes. In particular, cells that do not confer post-translational modifications that may be immunogenic, or cells with post-translational modifications that are immunologically identical to human, are most preferred. As is more fully described below, cell types implicated in a wide variety of disease conditions are particularly useful, so long as a suitable screen may be designed to allow the selection of cells that exhibit an altered phenotype as a consequence of the presence of a library member within the cell.

Accordingly, suitable mammalian, preferably human, cell types include, but are not limited to, tumor cells of all types (particularly melanoma, myeloid leukemia, carcinomas of the lung, breast, ovaries, colon, kidney, prostate, pancreas and testes) and cell lines derived from them (such as HeLa cells), cardiomyocytes, fibroblasts, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, adipocytes, neuronal cells, Schwanoma cell lines, and other endocrine and exocrine cells. Suitable cells also include known research cells, including, but not limited to, Jurkat T cells, NIH3T3 cells, human embryonic kidney (293) cells, Baby Hamster Kidney (BHK) cells, Chinese Hamster Ovary (CHO) cells, African green monkey kidney cells (COS cells), etc. See the ATCC cell line catalog, hereby expressly incorporated by reference.

Mammalian expression systems and vectors useful for expression are also known in the art, and include retroviral systems. A mammalian promoter is any DNA sequence capable of binding mammalian RNA polymerase and initiating the downstream (3′) transcription of a coding sequence for library protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

Insect cell systems. In one embodiment, library proteins are produced in insect cells. Drosophila melanogaster cells, Spodoptera frugiperda cells (SF9), are often used as host cells, among others. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994). Expression vectors are introduced into cultured insect cells using calcium phosphate transfection, liposome transfection, viral infection and other means analogous to the means available to mammalian cells.

Yeast and other eukaryotic microbial systems. In one embodiment, library proteins may be produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In addition, fungal species such as members of the genus Neurospora may be used for protein expression.

Bacterial systems. In another embodiment, library proteins are expressed in bacterial systems. Bacterial expression systems are well known in the art. E. coli, Bacillus subtilis, Streptococcus cremoris, and Streptococcus lividans are some of the known and useful bacteria for protein expression with established expression vectors readily available. A bacterial expression vector is usually a plasmid, and comprises a promoter, an efficient ribosome binding site, a coding region with a start codon and a stop codon, a transcription termination site, a selectable marker, and an origin of replication. The vector optionally comprises a signal peptide sequence after the start codon to direct the expressed protein into the growth media (gram-positive bacteria) or into the periplasmic space, located between the inner and outer membrane of the cell (gram-negative bacteria).

The bacterial expression vectors may be transformed into bacterial host cells using techniques well known in the art, such as calcium chloride treatment, electroporation, and others.

Selection and Screening of Expressed Proteins

Once expressed, novel proteins may be isolated or purified. The degree of purification necessary will vary depending on the use of the library protein and the method of assaying or screening. In general, the library proteins may be screened for one or more biological activities. These screens will be based on the scaffold protein chosen, as is known in the art. Thus, any number of protein activities or attributes may be tested, including binding to a known binding partner (for example, a substrate, ligand, co-factor, antibody, etc.), activity profiles, stability profiles (pH, thermal, buffer conditions), substrate specificity, immunogenicity, toxicity, etc. If purification is desired, a variety of suitable purification methods are known to those skilled in the art which may be selected depending on the composition of the sample. Standard purification methods include electrophoretic, molecular, immunological and chromatographic techniques, including ion exchange, hydrophobic, affinity, and reverse-phase HPLC chromatography, and chromatofocusing. For example, the library protein may be purified using a standard affinity column, such as antibody based column. Ultrafiltration and diafiltration techniques, in conjunction with protein concentration, are also useful. For general guidance in suitable purification techniques, see Scopes, R., Protein Purification, Springer-Verlag, NY (1982).

Alternatively, in some instances no purification may be necessary. For example, the screening may be carried out by looking for an altered phenotype of cells expressing the novel protein. The altered phenotype is due to the presence of a library member with a desired characteristic. By “altered phenotype” or “changed physiology” or other grammatical equivalents herein is meant that the phenotype of the cell is altered in some way, preferably in some detectable and/or measurable way. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in phosphorylation; changes in the secretion of ions, cytokines, hormones, growth factors, or other molecules; alterations in cellular membrane potentials, polarization, integrity or transport; changes in infectivity, susceptibility, latency, adhesion, and uptake of viruses and bacterial pathogens; etc. By “capable of altering the phenotype” herein is meant that the library member can change the phenotype of the cell in some detectable and/or measurable way.

The altered phenotype may be detected in a wide variety of ways, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death arising from virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc. In some cases, as is more fully described herein, the altered phenotype is detected in the cell in which the novel protein is expressed; in other embodiments, the altered phenotype is detected in a second cell which is responding to some molecular signal from the first cell.

5. Novel Proteins and their Uses

In another aspect, the invention provides a protein designed using the peptide sequence libraries described herein and selected from a protein library using the methods described above. The designed protein may be produced by any means known in the art, including peptide synthesis or by expression from recombinant DNA encoding the desired protein. Any of the expression systems and vectors described above can be used to produce a selected protein. In exemplary embodiments, a designed protein may be non-immunogenic, or have low immunogenicity, in humans. For example, a designed protein comprising at least one human peptide may have a reduced immunogenicity in comparison to a starting reference protein which did not contain any human peptide sequences. In one embodiment, the designed protein has no posttranslational modifications. This may be accomplished by designing protein sequences lacking the undesirable modification site; by using expression cells lacking the ability for undesirable posttranslational modifications either by nature or by genetic manipulation of the cells (i.e. deleting or altering certain necessary enzymes); and/or removing the undesirable modification (e.g. deglycosylation, deacetylation, etc.) after the protein is expressed and purified. In another embodiment, the designed protein is modified in a way that is not immunogenic in humans, for example, by comprising posttranslational modifications that are naturally found in humans.

Once isolated and purified to a degree and quality acceptable for therapeutic uses, the novel protein created, selected, and manufactured by the methods described herein may be administered to a human for a therapeutic purpose. As has been described herein, the novel protein will have little or no immunogenicity in humans, and one or more desired therapeutic characteristics, for example, the desired therapeutic activity, bioavailability, suitable in vivo stability and degradability, suitable targeting ability, or solubility, or any of the qualities that defines the term “characteristic” herein. A protein of the present invention is non-immunogenic because it will be processed for antigen presentation into peptides that are naturally found in humans so that tolerance to these peptides has been developed, e.g., the peptides are recognized as self by the human immune system.

A novel protein of the present invention may be based on a scaffold protein including, but not limited to, one of the scaffold proteins described above herein. The novel protein may therefore share a biological activity and/or one or more structural elements with the scaffold protein.

In another aspect, the invention provides a pharmaceutical composition comprising at least one novel protein as described herein. The composition may further comprise a pharmaceutically acceptable carrier and/or excipient. One exemplary pharmaceutically acceptable carrier is physiological saline. Other pharmaceutically acceptable carriers and their formulations are well-known and generally described in, for example, Remington's Pharmaceutical Science (18^{th} Ed., ed. Gennaro, Mack Publishing Co., Easton, Pa., 1990). Various pharmaceutically acceptable excipients are well-known in the art and can be found in, for example, Handbook of Pharmaceutical Excipients (4^{th} ed., Ed. Rowe et al. Pharmaceutical Press, Washington, D.C.). The pharmaceutical composition may be formulated for various routes of administration, including but not limited to oral, intravenous, intramuscular, subcutaneous, transdermal, pulmonary or intraperitoneal administration. The composition can be formulated as a solution, microemulsion, liposome, capsule, tablet, or other forms suitable for various routes of administration described above in for the methods of treatment. The active component which comprises the novel protein may be coated in a material to protect it from inactivation by the environment prior to reaching the target site of action. In another embodiment, the pharmaceutical composition is suitable for sustained release of the active ingredients, the composition comprising biologically compatible polymers or matrices that allow slow release of the therapeutically active novel protein. Such sustained release formulations may be in the form of, for example, transdermal patches, implants, or suppositories.

In another embodiment, the invention provides biochips comprising libraries of novel, engineered proteins, with the library comprising at least about 100 different proteins, with at least about 500 different proteins being preferred, about 1,000 different proteins being particularly preferred and about 5,000-10,000 being especially preferred. These proteins may then be screened again for a characteristic of interest.

### INCORPORATION BY REFERENCE

All of the patents, publications and sequence database entries cited herein are hereby incorporated by reference. Also incorporated by reference are the following: U.S. Patent Application Publication Nos: 2004/0259146; 2004/0241701; 2003/0096307; 2004/0043430; 2003/0036854; 2004/0152872; and 2002/0177691.

### EQUIVALENTS

Those skilled in the art will recognize, or be able to ascertain using no more than routine experimentation, many equivalents to the specific embodiments of the invention described herein. Such equivalents are intended to be encompassed by the following claims. Although the descriptions are for designing proteins that are non-immunogenic to humans, the same principle applies to designing proteins that are non-immunogenic to other vertebrates, including mammals such as mouse, rat, rabbit, dog, cat, horse, bovine, sheep, pig, or monkey.

What is claimed:

1. A library of sequences of peptide motifs found in human proteins, comprising a set of all sequences of human peptides having more than 4 amino acid residues, and less than about 50 amino acid residues.

2. The library of claim 1, wherein the library comprises sequences of peptides having about 6 to 15 amino acid residues.

3. The library of claim 1, further comprising information about the structure and conformations that the peptides may assume, and optionally additional information regarding the conformation.

4. The library of claim 1, wherein the peptide motifs are generated using proteasome or acid protease cleavage sites as the cleavage sites of the peptide sequences from naturally occurring human proteins.

5. The library of claim 1, wherein the peptide motifs are those of peptides presented by the Major Histocompatibility Complex I or II on the surface of human immune cells.

6. A library of sequences of peptide motifs found in human proteins, wherein the human proteins are members of a distinct class of molecules, said class defined by a structural motif or function.

7. A library comprising isolated polynucleotides encoding a set of all human peptide sequences having more than 4 amino acid residues, and less than about 50 amino acid residues.

8. A library comprising polynucleotides encoding peptide motifs found in human proteins, wherein the human proteins are members of a distinct class of molecules, said class defined by a structural motif or a function.

9. A method of designing a novel protein comprising:
(a) selecting a scaffold protein;
(b) identifying a partial structure of the scaffold protein to be replaced;
(c) computationally searching and identifying a human peptide, wherein the human peptide:
(i) is a member of a library comprising a set of all sequences of human peptides having more than 4 amino acid residues and less than about 50 amino acid residues; and
(ii) shares a structural motif with the partial structure of the scaffold protein;
(d) replacing a portion of the amino acid sequence of the scaffold protein corresponding to the partial structure with the amino acid sequence of the human peptide to produce a novel protein; and
(e) optimizing the structure of the novel protein to retain the structural motif.

10. A method of producing a novel protein, comprising:
(a) selecting a scaffold protein;
(b) identifying a partial structure of the scaffold protein to be replaced;
(c) computationally searching and identifying one or more human peptides, wherein the human peptides:
(i) are a member of library comprising a set of all sequences of human peptides having more than 4 amino acid residues and less than about 50 amino acid residues; and
(ii) share a structural motif with the partial structure; and
(d) replacing the partial structure sequence with the sequence of a human peptide to create a sequence of the novel protein;
(e) creating a polynucleotide that encodes the amino acid sequence of the novel protein; and
(f) expressing the polynucleotide to produce the novel protein.

11. A library of novel proteins, wherein the novel proteins are produced by the method of claim 10, and wherein the novel proteins are non-immunogenic in humans.

12. A method for producing a therapeutic, non-immunogenic protein comprising screening the library of claim 11 to identify a protein exhibiting a desired characteristic.

13. A protein which is non-immunogenic to humans, wherein the protein comprises human peptide segments, which peptide segments are recognized as self by the human immune system, and wherein the protein does not naturally occur in humans.

14. A protein produced by the method of claim 10.

15. A pharmaceutical composition comprising:
(a) an isolated and purified protein comprising human peptide segments, which peptide segments are recognized as self by the human immune system, and wherein the protein does not naturally occur in humans; and
(b) a pharmaceutically acceptable excipient.

16. A method of designing a novel protein comprising:
(a) selecting a scaffold protein;
(b) identifying a partial structure or disordered region of the scaffold protein to be replaced;
(c) computationally searching and identifying one or more human peptides, wherein the human peptides:
(i) are a member of a library comprising a set of all sequences of human peptides having more than 4 amino acid residues and less than about 50 amino acid residues; and
(ii) share a structural motif with the partial structure of the scaffold protein or are disordered;
(d) replacing a portion of the amino acid sequence of the scaffold protein corresponding to the partial structure or disordered region with the amino acid sequence of a human peptide to produce a novel protein; and
(e) optimizing the structure of the novel protein to retain the overall structure of the scaffold protein.

17. The method of claim 16, further comprising creating a polynucleotide that encodes the amino acid sequence of the novel protein.

18. The method of claim 17, further comprising expressing the polynucleotide to produce the novel protein.

19. The method of claim 16, wherein the novel protein is non-immunogenic in humans.

20. A library of novel proteins, wherein the novel proteins are produced by the method of claim 16.

21. A method for producing a therapeutic, non-immunogenic protein comprising screening the library of claim 20 to identify a protein exhibiting a desired characteristic.
